(19) United States
(12) Patent Application Publication
(10) Pub. No.: US 2023/0157578 A1
(43) Pub. Date: May 25, 2023

(54) ANALYSIS SYSTEM AND METHOD FOR ANALYZING THE MOVEMENT OF A PORTION OF THE BODY AND/OR OF A HUMAN PROSTHESIS

(71) Applicant: ECOLE POLYTECHNIQUE FEDERALE DE LAUSANNE EPFL-TTO, Lausanne (CH)

(72) Inventors: Andrea CREMA, Lausanne (CH); Fiorenzo ARTONI, Lausanne (CH); Silvestro MICERA, Lausanne (CH)

(73) Assignee: ECOLE POLYTECHNIQUE FEDERALE DE LAUSANNE EPFL-TTO, Lausanne (CH)

(21) Appl. No.: 17/920,371

(22) PCT Filed: Apr. 21, 2020
(86) PCT No.: PCT/EP2020/061072

§ 371 Date: Oct. 20, 2022

(51) Int. Cl. A61B 5/103; A61B 5/11; G01L 25/00; G16H 20/30

(52) U.S. Cl. CPC A61B 5/1038; A61B 5/112; G01L 25/00; G16H 20/30

(57) ABSTRACT

An analysis method for analyzing pressure exerted by a body portion and/or of a human prosthesis includes:
sampling data generated by the body portion and/or the prosthesis during a threshold calibration period at a sampling frequency in a condition of use thereby obtaining an array of sampled data of at least one sensing unit;
providing at least one threshold value of the sampled data for the condition of use and storing the least one threshold value in a memory unit;
comparing at the sampling frequency the sampled data with the at least one threshold value to obtain calibrated data, and
storing the calibrated data in the memory unit and/or sending the calibrated data to a computing device if the calibrated data is greater than the at least one threshold value. A sensing device for sensing the pressure exerted by a body portion and/or the human prosthesis is also provided.

### FIELD OF THE INVENTION

The present invention relates to an analysis system that senses and analyses the movement of a portion of the body and/or of a human prosthesis. The system of the invention is configured for sensing the movement of a portion of the body and/or of a human prosthesis and for processing the data emitted furnishing an analysis of the movement of the user. The invention further relates to a method for sensing and analyzing the movement of a portion of the body and/or of a human prosthesis.

The invention relates in particular to the field of wearable analysis systems and more particularly to an insole sensing system for monitoring foot or prosthesis contact with the insole surface. The insole sensing system is arranged for measuring the ground reaction force during movement. The human walking is analyzed through the gait analysis by collecting kinematic data and it is used in various fields. It is, for example, used for assessing gait pathologies, for monitoring rehabilitation outcomes and treat/prevent pressure ulcers in diabetes. In sport activities the gait analysis is used for optimizing the training performance, minimizing the risk of injury. The gait analysis is also important for the manufacturing of special footwear, e.g., for movement impaired persons or to minimize the risk of fall in the elders. Gait analysis is also important in the control of prostheses.

### BACKGROUND

Have been already proposed many systems for motion tracking, for example camera-based devices e.g. Vicon, Optitrack or Microsoft Kinect. These devices have a very high spatial precision but lack portability and are therefore unsuitable for applications outside laboratory settings. Motion tracking can also be performed via Inertial-Motion-Unit—IMU-based devices, such as the device described in “Xsens MVN: full 6DOF human motion tracking using miniature inertial sensors” Xsens Motion Technologies BV, Tech. Rep, vol. 1, 2009, D. Roetenberg, H. Luinge, and P. Slycke.

These devices deliver many information, however they all require calibration and complex sensor fusion algorithms to account for drifts and other noise and to reconstruct the kinematic model, “Concurrent validation of Xsens MVN measurement of lower limb joint angular kinematics” Physiological measurement, vol. 34, no. 8, J.-T. Zhang, A. C. Novak, B. Brouwer, and Q. Li, p. N63, 2013. These hardware and software constraints limit the maximum sampling rate achievable, which for instance for the Xsens, hovers around 60 Hz. Nevertheless, most of all in some applications, it is important to have a system which is portable and highly reactive. This is particularly important when the gait is analyzed in lower-limb amputees. The work “Intraneural stimulation elicits discrimination of textural features by artificial fingertip in intact and amputee humans,” C. M. Oddo et al., Elife, vol. 5, p. e09148, 2016′ showed that intraneural stimulation elicits discrimination of textural features by artificial fingertip in intact and amputee humans.

Using the same principle, it is possible to have a prosthetic foot contact trigger neuromorphic intraneural stimulation to provide feedback to the amputee on ground contact. To deliver this feedback without perceptible delay, assuming a trans-tibial amputation of 20 cm and considering an appropriate range of conduction velocity of peripheral nerves (medial/lateral plantar, tibial, saphenous), the response time of the device needs to be below 3 ms. It is also required that these systems are robust i.e. that they are able to furnish data and perform evaluation also in case of failure of one or more sensors.

U.S. Pat. No. 7,726,206 describes a foot pressure alert and sensing system comprising an insole provided with a plurality of pressure sensors placed in various points of the insole and operatively connected to a medical node which receives raw data from the nodes and generates pressure data and transmits pressure data to a base station. The insole is provided with primary and secondary sensors, a primary sensor is surrounded by secondary pressure sensors so that when the primary sensor is defective the primary sensor is activated. The number of secondary sensors provided for the primary sensor(s) is not necessarily the same throughout the insole.

Secondary sensors are cheaper than the primary sensors, as they are mainly intended to be activated only when one primary sensor is found defective. The medical node can receive data from primary and/or secondary sensors and can compensate for the differences in position between primary and secondary sensors.

The fault condition is recognized if the sensor returns the same raw data for a predetermined period of time or if it returns wildly erratic data that varies from high to low in a short period of time or produces useless data. Nevertheless, the system of U.S. Pat. No. 7,726,206 is not so robust, as it is not able to furnish correct information in case of multiple faults. The system of U.S. Pat. No. 7,726,206 does not provide for any check on the secondary sensors. Moreover, the detection obtained with the secondary sensors is different and even more less precise than the detection obtained with the primary sensors, as the secondary sensors are not the same as the primary sensors. Additionally, the system of U.S. Pat. No. 7,726,206 does not take into account the real activity performed by the user and, therefore, the indication about the condition of a sensor as faulty/working is not reliable. It is also known a system for plantar pressures and gait spatial-temporal parameters analysis available at the website http://www.technoconceptfr/shop/gait-rehabilitation/26-http-wwwtechnoconceptfr-shop-productphpidproduct26.html. The system, known as DYNAFOOT© is a system composed of insoles equipped with pressure sensors on its entire surface, arranged for recording the feet's load distribution by inserting them directly into the patient's shoe. The system further comprises a recording unit connected to the insole. The DYNAFOOT© is not suitable for timely control of robotic devices and for reliable estimation of brain/muscle connectivity.

A drawback of known system is that they have either a high sampling time, or, on the contrary, the data obtained by the systems are not accurate. The data obtained with the insole of DYNAFOOT© have noise and other disturbances and are likely to contaminate the output data. In order to achieve reliable data known systems thus rely on post processing filtering steps performed in a PC or similar computing device. This increases the communication delay. Therefore, data are available to the user for any evaluation after a considerable delay that impedes the possibility of using these data in certain applications in which only short delays are acceptable.

### SUMMARY OF THE INVENTION

Therefore, the need remains of a system for monitoring and/or analyzing the pressure applied from a part of the body of a user or from a human prosthesis attached to the body of a user that is portable and that at the same time furnishes reliable data about the sensed parameter. It is necessary to provide a wearable system that provides information about wear-out of sensors or damage due to use.

The scope of the invention is to provide an analysis system that at the same time provides accurate information about the pressure applied from a part of the body of a user or from a human prosthesis attached to the body of a user and that provides this information with a very short delay. A scope of the invention is to provide a system that is reliable as for the data measured, sampled and recorded.

Another scope of the invention is to provide a system that is suitable for furnishing reliable data and that is reactive, i.e. that provides reliable data in a short time.

A further object of the invention is to provide an analysis system that is able to perform gait segmentation and detailed analysis and classification of the contact phase during walking, running and other tasks.

It is another object of the invention to provide an analysis system that assesses the gait capabilities of humans in healthy conditions or with motor deficits, is able to quantify the spatial distribution of loads and the temporal patterns of loading and unloading of the foot, while minimizing the disturbances under the insole and allowing to record the response of the subject in relation to external triggered events or as a stream of data.

The response of the user is expressed as a motion response or in terms of pressure applied by the user.

According to an aspect of the invention it is provided an analysis method for analyzing the pressure exerted by a portion of the body and/or of a human prosthesis comprising the following steps:

sampling data generated by the portion of the body and/or the human prosthesis during a threshold calibration period at a sampling frequency in at least one condition of use so as to obtain an array of sampled data of at least one sensing unit;

providing at least one threshold value of the sampled data for the at least one condition of use and storing the at least one threshold value in a memory unit;

comparing the sampled data with the at least one threshold value to obtain calibrated data;

storing the calibrated data in the memory unit and/or send the calibrated data to a computing device if calibrated data are greater than at least one threshold value.

It has to be understood that physical quantities are “measured” by sensing units that transduce physical quantities (pressure for example) into electric signals that are emitted by the sensing units and then digitized i.e. quantized and sampled and then stored or processed, as will be explained in the following.

Owing to this aspect of the invention it is possible to store and use only useful data. If the calibrated values are lower than the at least one threshold value then they are discarded and not saved or transmitted. It is therefore, possible to save memory and/or to reduce the quantity of exchanged data and, therefore, to increase the responsiveness of the method of the invention.

It is also possible to provide only useful data for future evaluation. Therefore, the future evaluations are faster and at the same time more precise. The method of the invention is particularly responsive and owing to the method of the invention it is possible to reduce the response time required and to obtain information about the data sampled in a very short time. Additionally, owing to the method of the invention, it is possible to reduce the quantity of data stored and/or exchanged. It is also possible to obtain very reliable data in a very short time.

In the present specification with the term “reactive” it is intended a system that “reacts” to a change even minimal of the physical property to be measured.

In the present specification with the term “reliable” it is intended that the system provides a value void, as much as possible, of errors, in which the errors in the values provided are reduced and that the values provided reflect the change in the physical entity measured, i.e. provide a good indication about what really happened. It is also intended in the present specification with the definition “short time” an interval of time ≤2 ms.

Owing to the method of the invention it is also possible to reduce the noise affecting the emitted data.

Preferably, during use the data generated by the portion of the body are sampled a plurality of times at the sampling frequency at different sampling times. The data are processed preferably at the same frequency at which they are sampled, i.e. the method has a processing frequency that is preferably equal to the sampling frequency.

In a version the method provides for an evaluation phase that processes the sampled data to obtain calibrated data.

In a further version the method provides an operating process in which the calibrated data are processed to obtain processed data, the operating process comprising at least one the following steps: a fault-check process that processes calibrated data to obtain fault check data, an adaptation process that processes calibrated data or fault-check data to obtain adapted data, a gait segmentation process that processes calibrated data or fault-check data or adapted data to obtain the validated gait phase data.

The data obtained from the operating process are indicated as processed data.

In a preferred version the operating process comprises a fault-check process that processes calibrated data to obtain fault-check data, an adaptation process that processes fault-check data to obtain adapted data, a gait segmentation process that processes adapted data to obtain the validated gait phase data.

In the preferred condition the processed data are the adapted data which are sent on along with the validated gait phase data. Owing to the method of the invention at the end of the operating process there are obtained processed data and/or validated gait phase data that are stored and/or sent to a computing device, i.e. those data which have overcome the processes for checking their reliability, as explained in the following, for example the evaluation process, and/or the fault check process and/or the adaptation process.

In an advantageous version the condition of use is the onload condition: it is defined as the condition in which the user is in contact with the at least one sensing unit and exerts a pressure on the at least one sensing unit and is still or is moving and performing a task like for example walking, running, jumping, etc.

In a further version the condition of use is an offload condition in which the portion of the body and/or of a human prosthesis does not exert any pressure.

Advantageously, the method is executed on a plurality of sensing units, each sensing unit providing sampled data from the emitted data at the sampling frequency.

Advantageously, each sensing unit provides a physical signal, emitted data, that corresponds to a voltage signal the value of which depends on the pressure exerted by the user on the sensing unit, from the emitted data sampled data are obtained by sampling the emitted data.

Advantageously, for each condition of use the at least one threshold value takes into account the “minimum value” emitted by each sensing unit during a threshold calibration period at which the sensing unit is considered active at the use condition.

Advantageously for each condition of use it is further provided a second threshold value taking into account the variability over the threshold calibration period of the data emitted by each sensing unit, as explained in the following.

In a version of the method it is provided at least one first threshold value and one second threshold value for each condition of use of a plurality of conditions of use, preferably at least one first offload threshold value and one second offload threshold value for an offload condition, and/or at least one first onload threshold value and one second onload threshold value for an onload condition. This increases the precision of the method of the invention.

In a version, there is provided a set of first onload threshold values for the onload condition, each first onload threshold value of the set of first onload thresholds values taking into account different degrees of activation of the system on which the method is run. In this way, the activation of the sensing unit, or of the plurality of sensing units, is divided in many discrete intervals, corresponding to different degrees of activation. This allows to reduce the quantity of data exchanged and/or stored and to obtain at the same time useful information about the degree of activation of the different sensing units over time.

In a further version of the method it is provided a plurality of sets of onload threshold values, each set of onload threshold values corresponding to a different onload condition. The different onload conditions may be for example walking, running, obstacle racing, etc. The different sets of onload threshold values may have a preset number of first onload threshold values, so that the activation of the sensing unit or of the plurality of sensing units is divided in a preset number of discrete intervals. The number of first onload threshold values may differ for the different onload conditions.

Advantageously, the method of the invention further comprises a fault check process which allows any fault of the system to be detected.

The fault check process allows the correct functioning of the sensing units of the system to be checked. The fault check process allows functioning and reliability of the data transmitted and/or stored to be guaranteed. This allows only reliable data to be taken into account. This also minimizes the risk that wrong data, obtained by non-working sensing unit(s), are taken into account, since this would jeopardize the final results.

In a version, the analysis method further comprises an adaptation process in which the method is adapted to the user on which the method is performed and/or to the condition of use. This allows to take into account the different characteristics of the user, for example different weight, etc., and/or the different conditions of use, for example walking, running, etc. This allows to adapt the method on the actual condition of use and/or to different users.

In a further version of the method it is provided for calculating the at least one threshold value. According to a preferred version, the method of the invention comprises a calibration process which is provided for calculating the at least one threshold value of the sampled data for the at least one condition of use. The calibration process comprises advantageously an offload calibration step and an onload calibration step. The offload calibration step comprises a first offload threshold calculation step in which a first offload threshold value is calculated and a second offload threshold calculation step in which a second offload threshold is calculated. Similarly, the onload calibration step comprises a first onload threshold calculation step in which at least a first onload threshold value is calculated and a second onload threshold calculation step in which a second onload threshold value is calculated.

In a version of the method it is provided a gait segmentation process in which it is provided for defining a plurality of gait phases during the task performed by the user to obtain reliable information on the gait phase and on the degree of activation of the sensing units in the different gait phases. The gait phases depend on the task to be performed and are usually cyclically repeated depending on the specific task. This allows for example to understand if the position and/or loading of the feet or prosthesis is correct during the different gait phases and/or to detect if there are some problems or load asymmetries during the different gait phases. In this way, it is possible to correct any problems if desired.

In a version of the method in the gait segmentation process it is provided for emitting information connected to the gait phase. In this case the gait segmentation process is provided for comparing the calibrated data, or fault-check data or adapted data with weight matrices to decide about the gait phase and to emit a corresponding signal to the computing device.

In an advantageous version the method is performed in an acquisition device connected with a user.

According to another aspect of the invention it is provided a sensing device for sensing the pressure exerted by the portion of the body and/or the human prosthesis, comprising a body formed by a flexible material and having a plurality of superimposed layers, each layer of the plurality of layers being provided with a plurality of sensing units configured for sensing the pressure applied by the portion of the body of the user and/or the human prosthesis, the sensing units of the plurality of sensing units being a capacitive or resistive/capacitive force/pressure sensing unit.

In a version, the sensing units of the different layers are positioned in corresponding positions in the body of the sensing device. In a preferred version the sensing device is an insole, or a glove.

Advantageously, the sensing units of the different layers are positioned in the body of the sensing device in positions corresponding to the more sensitive areas, i.e. in the areas in which the pressure exerted by the user is likely to change more frequently and/or it is likely higher than in other areas.

According to a further aspect of the invention it is provided an acquisition device for an analysis system arranged for analyzing the pressure exerted by a portion of the body and/or of a human prosthesis, the acquisition device comprising: connection means for connecting the acquisition device to at least one sensing unit arranged for being positioned in contact with the portion of the body and/or the human prosthesis to be analyzed and configured for measuring generated data generated by the portion of the body and/or the human prosthesis, and for transferring generated data to the acquisition device, the acquisition device comprising a processing device for sampling data generated by the portion of the body and/or the human prosthesis during a threshold calibration period at a sampling frequency in at least one condition of use so as to obtain an array of sampled data of the at least one sensing unit, a memory unit in which at least one threshold value of the sampled data for the at least one condition of use is stored and the processing device being further suitable for comparing the sampled data with the at least one threshold value to obtain calibrated data and to store the calibrated data in a memory unit and/or to send calibrated data to a computing device if they are greater than the stored at least one threshold values.

According to another aspect of the invention it is provided an analysis system for analyzing the pressure exerted by a portion of the body and/or of a human prosthesis comprising: a sensing device, an acquisition device operatively connected to the sensing device through suitable connection means so as to receive generated data from the sensing device, a computing device operatively connected to the acquisition device through further connection means and arranged for receiving generated data from the acquisition device, the acquisition device comprising a memory unit in which at least one threshold value of the generated data is stored and a processing device for sampling sampled data from the emitted data from the sensing device and for processing the sampled data to obtain calibrated data and/or processed data which are stored in the memory unit or sent to the computing device.

The system of the invention is very reactive, as it is capable of furnishing reliable data in short time.

Owing to the invention it is possible to process data emitted by the sensing device and storing data in the acquisition device or sending them to a computing device for subsequent evaluation only if the data values are equal to or greater than the preset at least one threshold data. In this way, useless data are not stored in the acquisition device nor sent to the computing device. This implies that the space occupied in the memory of the acquisition device is reduced and a greater quantity of useful data is stored. This further implies that the quantity of data sent to the computing device is reduced and, therefore, that the analysis time is greatly reduced and the system is more effective and allows a faster and more realistic analysis to be obtained.

This also allows the response time of the computing device to be reduced, i.e. the moment in which the data are available at the computing device is very close to the moment in which they have been emitted, sampled and sent.

This also allows to store in the memory of the acquisition device only useful data which can be readily used by the user and which provide useful information about the movement of a portion of the body and/or of a human prosthesis.

Additionally, the system of the invention allows noise and other disturbances which could contaminate the output data furnished by the acquisition device to the computing device to be extremely reduced.

Usually, the data sent by the acquisition device are processed in a computer in order to filter them and eliminate noise and errors, these processing steps being made in the acquisition device of the system of the invention.

The analysis system of the invention is fault tolerant, since it automatically detects faults as well as drifts and degradation of performance and it is responsive since the delay detection of pressure variation is very low, light.

Additionally, the analysis system of the invention allows real-time gait segmentation, and wireless communication. The system of the invention is able to perform a reliable and precise gait segmentation and to furnish a response in a response time ≤10 ms, preferably ≤2 ms.

### BRIEF DESCRIPTION OF THE DRAWINGS

The features and advantages of the invention will be better appreciated from the detailed description of some preferred embodiments thereof, illustrated by way of non-limiting example with reference to the appended drawings, in which:

FIG. 1 is a schematic view of an analysis system of the invention;

FIG. 2 is a lateral exploded view of an insole according to the invention;

FIG. 3 is a front view of a layer of the insole of FIG. 2;

FIG. 4 is a schematic view of an acquisition device of the invention;

FIG. 5 is a perspective view of the acquisition device of FIG. 4;

FIGS. 6A and 6B are graphic representations indicating possible representations of the pressure of each sensing unit with respect to time of an insole in two conditions of use, respectively normal walk (FIG. 6A) and sprint (FIG. 6B);

FIG. 7 represents another graphic representation indicating a possible representation of the pressure of each sensing units with respect to time of two insoles of the same user.

FIG. 8 is a graphic representation of the synchronization between multiple devices of the method of the invention;

FIG. 9 is a flowchart of an analysis method of the invention;

FIGS. 10A-10C and FIGS. 11A-11C are diagrams illustrating the steps for the calculation of a first and second threshold value according to the invention;

FIGS. 12A-12C are diagrams illustrating some of the steps of the adaptation process according to the invention;

FIGS. 13A-130 are diagrams illustrating some of the steps of the fault check process according to the invention.

### DETAILED DESCRIPTION OF THE PREFERRED EMBODIMENTS

With reference to the FIGS. 1-5 it is shown an analysis system 100 according to the invention. The analysis system 100 is particularly suitable for sensing and analyzing the pressure exerted by a portion of the body and/or of a human prosthesis attached to the body of a user furnishing information about the position of a portion of the body and/or of a human prosthesis. The analysis system 100 of the invention is further configured for sensing the movement of the portion of the body and/or of a human prosthesis and for processing the sampled data furnishing an analysis of the movement. Therefore, the analysis system 100 is suitable for analyzing the pressure exerted by the portion of the body and/or of a human prosthesis either in a static or still (lack of movement) and in a dynamic condition (during movement). The analysis system 100 is particularly suitable for sensing the pressure applied by the foot of a user or by a prosthesis, in particular during walking or running, and for analyzing the sampled data furnishing information about the gait movement. Nevertheless, the analysis system 100 of the invention could also be used for sensing the pressure applied by any part of the body of the user or by any prosthesis of the user, for example in prosthetic gloves for upper limb amputee furnishing information about the movement thereof.

In other version of the invention, it is provided for sensing the force applied by the foot of a user or by a prosthesis during walking, or running, and analyses the sampled data furnishing information about the gait movement.

In general terms, the analysis system 100 of the invention comprises a sensing device 1 arranged for being positioned in contact with the portion of the body and/or the human prosthesis to be analyzed and configured for sensing the pressure applied by the portion of the body and/or the human prosthesis, and an acquisition device 2, operatively connected to the sensing device 1 through suitable connection means 3 so as to receive the pressure emitted data from the sensing device 1. The acquisition device 2 is arranged for receiving physical data from the sensing device 1 through the connection means 3 and amplifies and samples the transduced physical modifications generated by the sensing device 1, and converts this data on sampled data that can then be transmitted to the computing device 4 through the further connection means 5 and/or stored in the acquisition device 2, as will be explained in detail in the following. The computing device 4 may be a smartphone, PC, tablet, laptop or any other computing device provided with a CPU, MCU or any device that can process and show data arranged for processing data received by the acquisition device 2 and it can be provided with a display unit 6 for showing the data emitted, sampled and processed. The shape of the sensing device 1 is chosen according to the portion of the body to be measured.

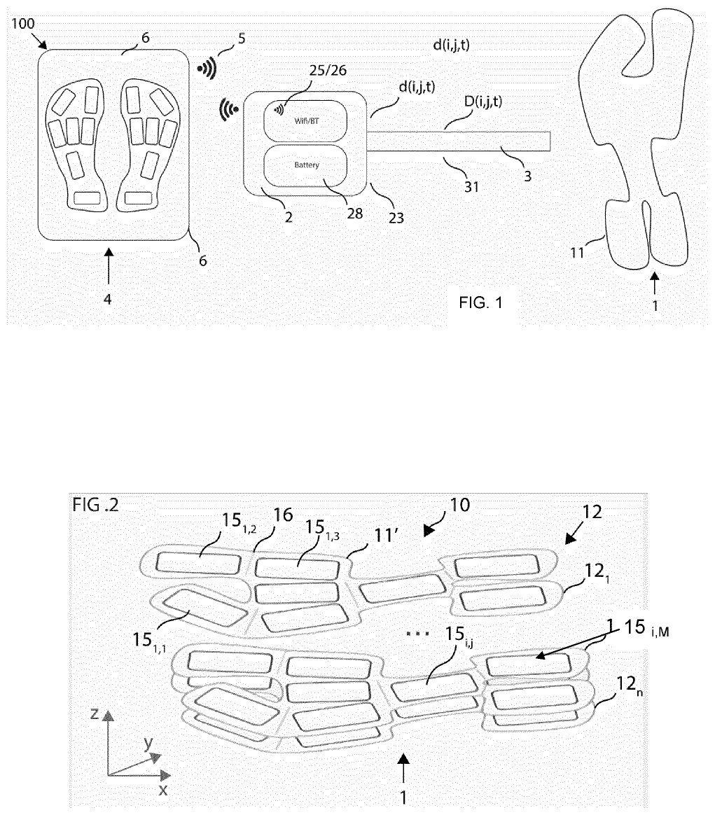

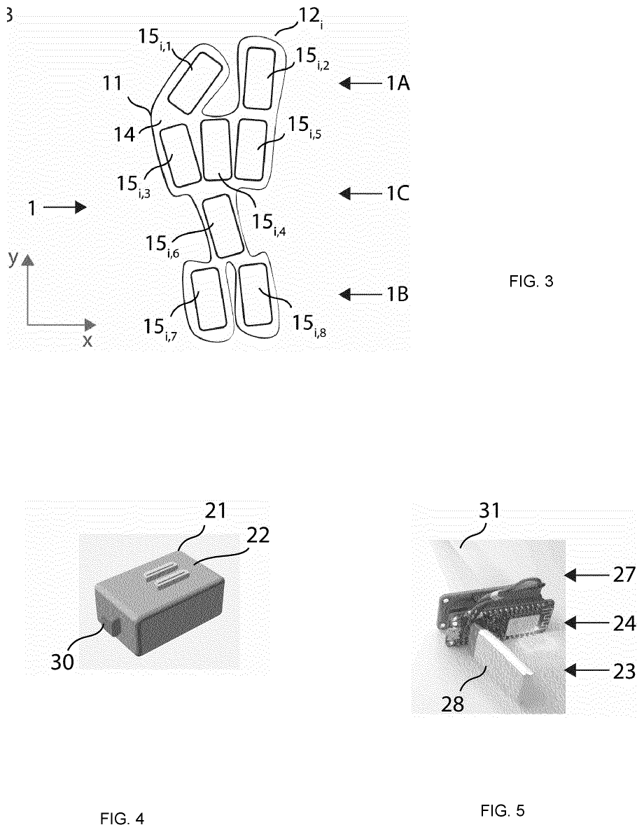

In the attached Figures an exemplary embodiment of the analysis system 100 is shown, many modifications can be carried out to the parts of the analysis system 100 without departing from the scope of the invention. In the version shown in the Figures the analysis system 100, shown in FIG. 1, comprises a sensing device 1 shaped as an insole 1, shown in greater detail in FIGS. 2 and 3, arranged to be positioned in contact with the foot, or the prosthetic foot, for sensing the pressure applied by the foot or the prosthetic foot, an acquisition device 2, shown in greater detail in FIGS. 4 and 5, operatively connected to the sensing device 1 through connection means 3 so as to receive emitted data from the sensing device 1, and a computing device 4 operatively connected to the acquisition device 2 by means of further connection means 5. The acquisition device 2 is arranged for receiving emitted data from the sensing device 1 and to sample and process these data so as to obtain processed data as explained in the following. The processed data can be stored in the acquisition device 2 and/or sent to the computing device 4 by means of the further connection means 5, as will be explained in detail in the following.

The acquisition device 2 processes also the sampled data to obtain processed data that can be stored in the acquisition device 2 and/or sent to the computing device 4. In a version, the acquisition device 2 also makes advanced processing of data so as to obtain processed data, in this case, the computing device can also not be provided, as explained below.

Even if in the following reference will be made to an analysis system 100 for sensing the pressure applied to the foot of the user, it should be understood that the system may be also used with a foot prosthesis. Additionally, it should be understood that the analysis system 100 of the invention could be used for sensing the pressure applied by any part of the body of the user, in particular the hand of the user and/or the hand prosthesis. Additionally, even if reference will be made in the description to an analysis system 100 for sensing the pressure, it should be understood that the system could also be arranged for sensing the force or weight any other entity causing a variation in the sensor of the analysis system 100.

With reference to FIG. 2, in a Cartesian coordinate system (chosen here for convenience) the length X of the insole 1 is measured along the longitudinal axis X, the width along the transversal axis Y and the thickness of the insole 1 is measured along the vertical axis Z. The insole 1 comprises a body 11 formed by a flexible material and provided with a plurality of sensing units 10 arranged into the body 11 and configured for sensing the force applied by the foot of the user. Each sensing unit of the plurality of sensing units 10 may be a resistive/capacitive force/pressure sensing unit, and modifies its properties based on the pressure it is subjected to, as explained in the following. The insole 1 is arranged to be positioned in a footwear, therefore, the body 11 may be shaped so as to resemble the shape of the sole of a footwear. In some versions, the body 11 is so shaped to form only a portion of an insole. In the preferred embodiment the insole 1 is so shaped to absorb the load and to conform to uneven loads and contact surfaces. The insole 1 comprises a plurality of insole layers 12, which are better visible in FIG. 2, superimposed one over the other along the thickness, i.e. along the vertical axis Z, of the insole 1 and mutually joined one to another for example by means of an adhesive so as to obtain a single insole 1. The generic insole layer is indicated as 12ᵢ where i=(1, . . . , N) indicates the i^{th} insole layer in the insole 1. The number of insole layers N of the insole 1 may be chosen during manufacturing of the insole 1: increasing the number of insole layers N of the insole layers 12 of the insole 1, the reliability and robustness of the analysis system 100 of the invention is increased, as will be explained in further detail in the following. The number of insole layers N is usually comprised between 2 and 20, preferably about 3-10.

As better visible in FIG. 3, each insole layer 12ᵢ comprises a body portion 11′ having the shape of the insole 1 and a one or more sensing units 15_{i,1}-15_{i,M} suitably spaced in the body portion 11′. The sensing units of insole layer 12ᵢ, 15_{i,j}, (j=1, . . . M) are placed along the length Y and width X of the insole 1 and are so positioned to define in the body portion 11′ different inert regions 14 spacing the sensing units 15_{i,j} (j=1, . . . M) of the insole layer 12ᵢ. In this way, the sensing units 15_{i,j} (j=1, . . . M) of insole layer 12ᵢ are surrounded by at least one inert region 14 made of flexible material. It should be noted that with the term “inert” it is intended in the present case regions void of sensing units. Each insole layer 12ᵢ is so shaped that deformations and bending of the insole 1 occur mainly at the inert regions 14. This will increase the reliability of the insole 1 of the invention avoiding damages to the plurality of sensing units 10. At the inert regions 14 may be, therefore, identified folding lines 16 at which the insole 1 may be bent during use. Each insole layer 12ᵢ is designed in such a way that in case of bending, the flatness of each sensing unit 15_{j,j} is preserved, while distortions are limited to the inert region 14, i.e. to the non-sensing portion of the insole 1.

Each sensing unit 15_{i,j} can be composed of polylines, joined in a closed flat shape having a desired number of sides, or can also have a polygonal or round shape. The sensing units 15_{i,j} of the plurality of sensing units 10 are arranged into the body 11 of the insole 1 and are configured for sensing the force or the pressure applied by the foot of the user. Each sensing unit 15_{i,j} is operatively connected to the acquisition device 2 so that the data sensed by the plurality of sensing units 10 are transmitted to the acquisition device 2. Each sensing unit 15_{i,j} of the plurality of sensing units 10 may be a resistive/capacitive force/pressure sensing unit, and modifies its properties based on the pressure it is subjected to, as explained in the following. The number of sensing units in each layer 12ᵢ of the insole 1 may be chosen during the manufacturing of the insole 1. Each insole layer 12ᵢ comprises a preset number “M” of sensing units 15_{i,j}, j=(1, . . . M), suitably spaced in the insole layer 12ᵢ, wherein “M” is between 2 and 200, preferably between 2 and 50, more preferably between 3 and 12, most preferably about 8. The number M of sensing units 15_{i,j} in the insole layers 12ᵢ of the insole 1 is chosen during the manufacturing of the insole 1 in dependence of the accuracy requested and/or on the final use of the system of the invention. Usually, the number of sensing units 15_{i,j} for each insole layer 12ᵢ is preferably constant in an insole 1; nonetheless, in some cases some of the insole layers 12ᵢ may have a different number of sensing units 15_{i,j}. The generic sensing unit of the insole layer 12ᵢ is indicated as 15_{i,j} where j=(1, . . . M) indicates the number of sensing units in a layer and i=(1, . . . N) indicates the number of layers of the insole 1.

As indicated above, the number N of the plurality of insole layers 12 and the number M of sensing units 15_{i,j} per each insole layer 12ᵢ may be chosen during the manufacturing of the insole 1.

If the insole 1 has a number N of insole layers 12ᵢ and each insole layer 12ᵢ has a number M of sensing units, the insole 1 has N*M sensing units 15_{i,j}. Schematically, considering a matrix positioning of the sensing units 15_{i,j} in the insole 1, there are N sensing units along the vertical axis Z, i.e. having a corresponding position in the different layers 12₁-12_{N} and per each insole layer 12ᵢ there are M sensing units 15_{i,j}.

| | |
|---|---|
| 12₁: | 15_{1, 1} . . . 15_{1, M} |
| | . . . |
| 12ᵢ: | 15_{i, 1} . . . 15_{i, M} |
| | . . . |
| 12_{N}: | 15_{N, 1} . . . 15_{N, M} |

The sensing units 15_{i,j} of the different insole layers 12ᵢ are positioned preferably in mutually corresponding positions, as will be explained in further detail in the following.

According to the invention it is preferred that each insole layer 12ᵢ of the insole 1 is provided with at least one sensing unit 15_{i,j} in the toe part 1A of the insole 1, at least one sensing unit 15_{i,j} in the heel part 1B of the insole 1 and at least one sensing unit in the plantar part 10 of the insole 1. Preferably the toe part 1A of the insole 1 comprises at least one sensing unit at the hallux region and at least another sensing unit in correspondence of the remaining fingers; the heel part 1B comprises four sensing units at the opposite lateral edges thereof, i.e. preferably at least one at the internal side and preferably at least one at the external side of the heel part and the middle part 10 of the insole 1 comprises at least one sensing unit in correspondence of the arc of the foot and at least one sensing unit in correspondence of the metatarsal bearing region. It is indicated with internal side, the side of the insole facing during use the opposite foot.

In the version shown in FIGS. 2 and 3, for each insole layer 12ᵢ, the insole 1 comprises a first sensing unit 15_{i,1} at the hallux region, a second sensing unit 15_{i,2} in correspondence of the remaining fingers, a third 15_{i,3}, fourth 15_{i,4} and fifth sensing unit 15_{i,5} in correspondence of the metatarsal bearing region, a sixth sensing unit 15_{i,6} at the arc of the foot and a seventh sensing unit 15_{i,7} at the internal side of the heel part 1B, and eighth 15_{i,8} at the external side of the heel part.

The acquisition device 2 comprises connection means 3 for operatively connecting the acquisition device 2 to the insole 1, further connection means 5 for operatively connecting the acquisition device 2 to the computing device 4.

In the version shown the connection means 3 is a cable 31 connecting the insole 1 and the acquisition device 2 allowing the transfer of data from the insole 1 to the acquisition device 2. The further connection means 5 is preferably a wireless connection means, for example WiFi, Bluetooth or other electronic signals (e.g., TTL logic, DAC, serial or parallel protocols, etc.) The acquisition device 2 processes the data emitted by the insole 1, samples sampled data from the emitted data and processes the sampled data storing the processed data in a memory unit 24 of the acquisition device 2 and/or delivering the processed data to the computing device 4.

The acquisition device 2, better visible in FIGS. 4 and 5, comprises a case 22 provided with a fastening device 21 for removably fastening the acquisition device 2 to the footwear of the user. The case 22 is small and light so as not to impact on regular gait activity of the user. The configuration of the acquisition device 2 and of fastening device 21 allows the acquisition device 2 to be fastened to the desired footwear. The fastening device 21 allows the acquisition device 2 to be fastened on the footwear tongue, eyelet or throat line or above the ankle on the lateral side inside of the leg. These different mounting options are aimed at providing flexibility of positioning with different footwear, and are aimed at not impairing movement.

The acquisition device 2 further comprises a processing device 23 housed in the case 22 that is suitable for sampling the emitted data from the insole 1 and for processing the sampled data, as explained in the following, and a memory unit 24 for storing data in the acquisition device 2.

In some versions not shown, the acquisition device is provided with a plurality of memory units.

The processing device 23 is operatively connected to the memory unit 24 so that processed data processed by the processing device 23 may be stored in the memory unit 24.

The processing device 23 is operatively connectable to the computing device 4 so that the data processed from the processing device 23 may be sent to the computing device 4.

The processing device 23 performs low and mid-level processing operations based on the firmware of the processing device 23, as explained in the following. The type of the processing device 23 depends on the type of sensing units provided in the insole 1. The processing device 23 receives emitted physical data from the insole 1 and sample sampled data from the emitted data and processes the physical data retrieved by sampling them and converting them in quantized digital data that are transmitted to the computing device 4 and/or stored in the memory unit 24. In a version, the acquisition device is provided with one processing unit for signal conditioning e.g., hardware amplification, detrending, filtering etc., the type of these processing units being dependent on the type of sensors used in the insole 1 and a further processing unit for processing and sampling the signals.

In other versions of the acquisition device, not shown in the Figures, a different number of processing units may be provided. The acquisition device 2 is further provided with a first 25 and a second 26 I/O interface for connecting the acquisition device 2 to the insole 1 and to the computing device 4 respectively and with a removable memory 27 for local dump of raw and processed data to be retrieved at the end of the recording. The first and second I/O interfaces 25, 26 may comprise one or more connectors which can be different one from another.

The acquisition device 2 further comprises a battery 28 (and battery management unit, with or without energy harvesting devices) for energizing the acquisition device 2 and allowing the functioning thereof and an activation button 30 provided on the case 22 and arranged to be activated by a user for activating/deactivating the analysis system 100 of the invention, as will be explained in the following.

The computing device 4 comprises a CPU, MCU or any device that can show data and process data received by the acquisition device 2 and a display unit 6 for showing the data emitted and processed.

Owing to the system and the method of the invention, processed data and/or calibrated data are available on the memory unit 24 of the acquisition device 2 or on the computing device 4 and shown in the display unit of the computing device 4 very soon.

As soon as the acquisition device 2 is powered on, the acquisition device 2 receives emitted physical data from the insole 1, it transmits via suitable further connection means 5, i.e. wireless or wired connection, its ID to the computing device 4 and once secure communication is established the acquisition device 2 transmits raw data and/or processed data or calibrated data at the selected rate to the computing device 4. Therefore, the system provides immediately a feedback for the user of the sensing device, as explained in the following.

Owing to the system of the invention, it is possible to achieve both reliability and fast response, as explained in the following. Both the structure of the sensing device 1 and of the acquisition device 2 allow these objects to be obtained.

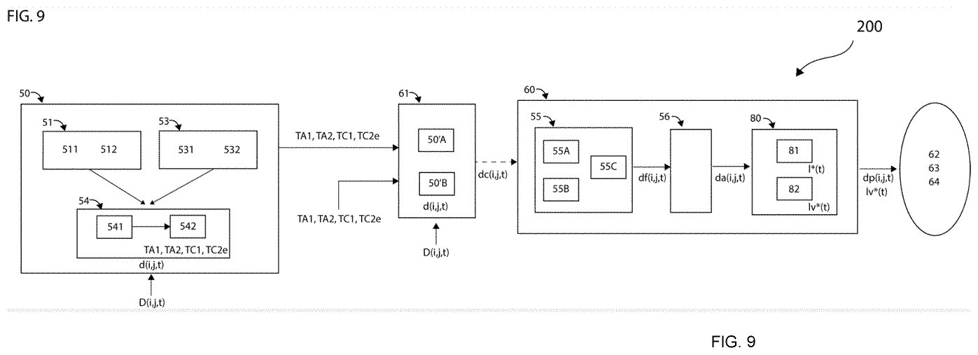

The analysis system 100 may operate according to an analysis method 200 comprising the steps explained below and indicated in the flowchart of FIG. 9.

The analysis method 200 comprises a calibration process 50 in which at least one threshold value of the analysis system 100 is calculated and stored in the memory unit 24 and an operating process 60 in which the analysis system 100 processes calibrated data dc(i,j,t) obtaining processed data dp(i,j,t) which are dependent on the functioning of the system, as explained in the following, and sends processed data dp(i,j,t) to the appropriate destination (e.g., memorization, display). The analysis method 200 further comprises an evaluation phase 61 in which sampled data d(i,j,t) are processed to obtain calibrated data dc(i,j,t) that are then sent to the operating process 60.

The calibration process 50 comprises advantageously an offload calibration step 51 and an onload calibration step 53. The offload calibration step 51 comprises a first offload threshold calculation step 511 in which a first threshold value is calculated and a second offload threshold calculation step 512 in which a second threshold is calculated. Similarly, the onload calibration step 53 comprises a first onload threshold calculation step 531 in which at least a first threshold value is calculated and a second onload threshold calculation step 532 in which a second threshold value is calculated.

In a version, the threshold values are preset in the analysis system 100 of the invention and stored in the memory unit 24 of the acquisition device 2, in this case the calibration process 50 is not run. In this case, in the evaluating phase 61 the preset threshold values stored in the memory unit 24 are used.

The calibration process 50 comprises a validation process 54 for validating the threshold values calculated in the threshold calculation steps 511, 512, 531, 532 to avoid detrimental effects due to noise or errors.

The operating process 60 of the analysis method 200 comprises a fault check process 55 which allows to detect faults of the analysis system 100 and produces the fault-check data df(i,j,t), as described in the following. In a version of the analysis method 200 not shown in the Figures the fault check process is run also during the calibration process 50.

In the fault check process 55 the correct functioning of the sensing units 15_{i,j} of the system is checked. The fault check process 55 is executed by the acquisition device 2 during the operating process 60 and uses the calibrated data dc(i,j,t) obtained from the evaluation phase 61 to check if one or more sensing units 15_{i,j} are faulty, as explained in the following. The fault check process 55 allows functioning and reliability of the data transmitted to the computing device 4 and/or stored in the memory unit 24 to be guaranteed. The fault check process 55 also allows a faulty condition to be immediately recognized and communicated.

The operating process 60 of the analysis method 200 further comprises an adaptation process 56 for adjusting the functioning of the analysis system 100 to the real conditions of use, i.e. to the actual user and/or to the actual task to be performed. There are several situations in which it is not possible or practical to easily remove the insole 1, for example if the insole is embedded in a prosthesis or because a fault occurs during use, especially in situations where it is not practical to replace sensors (e.g., during athletics competition) and it is important to furnish reliable data also in these cases. Furthermore, it is important to know, prior to beginning the activity, the status and efficiency of the insole. This objective is achieved owing to the adaptation process 56. The adaptation process 56 aims at adapting the analysis system 100 to the actual situation of use.

The adaptation process 56 is preferably run on fault check data df(i,j,t) or, failing that, on calibrated data dc(i,j,t). The following description refers to calibrated data dc(i,j,t) without loss of generality.

The adaptation process 56 allows a transfer function between applied weight and electric signal yielded by the acquisition device 2 to be calculated, so that the sampled data are adapted to the actual condition of use, owing to the adaptation process 56 calibrated data dc(i,j,t) are transformed into adapted data da(i,j,t) that can be used, instead of the calibrated data dc(i,j,t) in the operating process 60, as explained in the following.

The adaptation process 56 allows to take into account the modification of the condition of use to obtain processed data in a variety of conditions of use. There are several situations in which the load and, therefore, the pressure applied in all or some of the sensing units of the insole 1 is modified, for example because the insole is positioned in a different footwear and/or because the footwear is laced in tighter or looser way and/or because the user is changed, and it is very important to furnish reliable data also in these cases.

Furthermore, it is important to know, prior to beginning the activity, the status and efficiency of the insole.

The operating process 60 of the analysis method 200 further comprises a gait segmentation process 80 for analyzing the movement of the user in the different condition of use and for giving information about the gait phase. Owing to the analysis method of the invention the data emitted by the sensing unit may be processed to filter data coming from active or non-faulty sensing units, and/or to filter data which are at the noise level and/or processed to evaluate the level of activation of the sensing unit corresponding to the signal produced and/or the corresponding gait phase.

The gait segmentation process 80 processes the adapted data, or the fault-check data or the calibrated data to obtain validated gait phase data. In the following as an example, the gait segmentation process 80 is explained with reference to the adapted data da(i,j,t).

The analysis method 200 of the invention further comprises an evaluation phase 61 in which physical data from the insole 1 are sampled by the processing device 23 of the acquisition device 2. The evaluation phase 61 comprises a first and a second threshold comparing process 50′A, 50′B in which sampled data are compared with the threshold values obtained in the calibration process 50 and/or stored in the memory unit 24, as better explained in the following, and calibrated data dc(i,j,t) are emitted.

The operating process 60 receives data by the evaluation phase 61, and processes these data to obtain processed data. The processed data may be stored in a memory unit 24 through the memorization phase 62, and/or sent on the computing device 4 via the sending process 64 and possibly displayed. In some condition in the operating process 60 it is provided for emitting an alarm 63 based on the results of the operating process 60.

Owing to the analysis method 200 of the invention the produced values are responsive to the movement of the user which can be stored and/or visualized in the display unit 6.

The phases of the analysis method 200 are discussed below and explained with particular reference to FIGS. 9-14.

The analysis system 100 is firstly switched on and the all the sensing units 15_{i,j} of the plurality of sensing units 10 of the insole 1 are switched on and begin to detect the pressure applied thereon emitting continuous emitted data D(i,j,t) where “D” indicates data, i, j identify the sensing unit 15_{i,j} and “t” indicates the time. Emitted data D(i,j,t) are emitted as continuous electrical signals by the sensing units 15_{i,j}. The acquisition device 2 samples data from the emitted data D(i,j,t) at a sampling frequency Fs that is usually about 500-1000 Hz, so that the time interval between two consecutive data samples is usually about 1-2 ms, obtaining an array of sampled data indicated as d(i,j,t).

Therefore, only sampled data d(i,j,t) are used by the acquisition device 2.

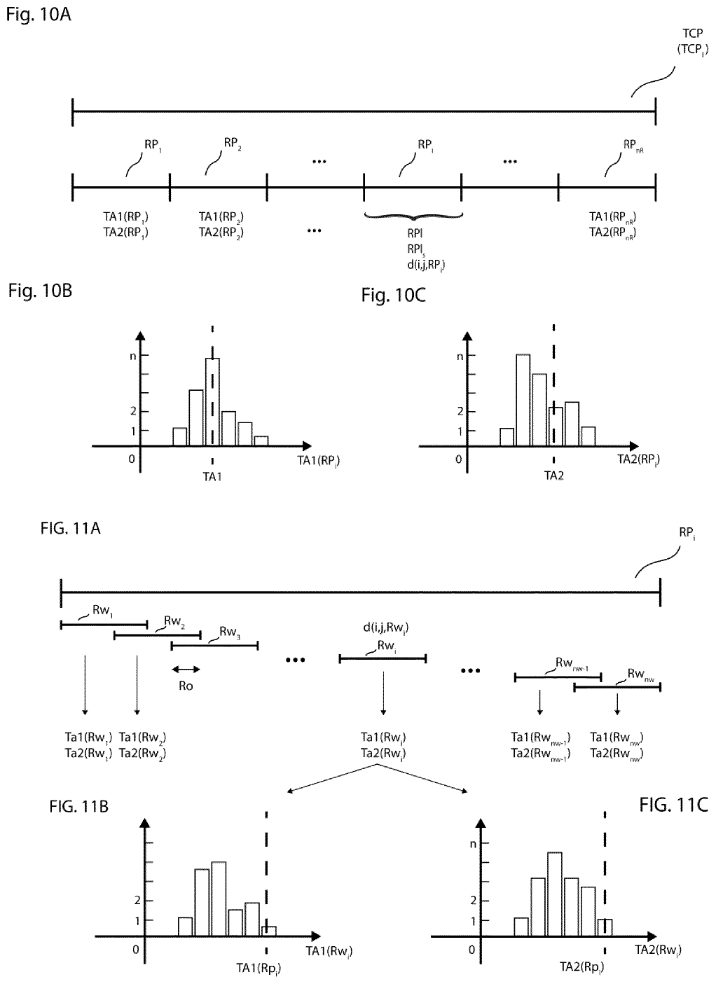

If it is provided, the calibration process 50 begins after having turned on the analysis system 100 by acting on the activation button 30 and allows the first initialization of the analysis system 100 of the invention to be accomplished. The calibration process 50 is advantageously made by connecting the computing device 4 to the acquisition device 2, and it is performed by the computing device 4. The calibration process 50 lasts for the threshold calibration period TCP lasting a threshold calibration period length TCPI (see FIG. 10A), generally between 10 and 300 seconds, preferably about 30-60 seconds and allows the threshold values of the sensing units 15_{i,j} for different operating conditions of the analysis system 100 to be calculated. The calibration process 50 comprises different calibration steps, each performed in a different condition of use.

In the version shown, the calibration process 50 comprises an offload calibration step 51 performed on the analysis system 100 while the user is not wearing the insole 1, and an onload calibration step 53 while the user is wearing the insole 1 and is performing a movement task, for example walking, running, etc.

In another version further calibration steps for different conditions of use could be provided, for example for different onload conditions each related to a different task.

In further versions the calibration process 50 comprises only an onload calibration step in which the onload threshold value/values is/are calculated.

In each of the above indicated calibration steps 51, 53, at least two different thresholds values are calculated by the analysis system 100, as will be explained below. The calculated threshold values are then stored in the memory unit 24 of the acquisition device 2.

At each calibration step 51, 53 of the calibration process 50 it is calculated a first threshold value that takes into account the amplitude distribution of the sampled data d(i,j,t) by each sensing unit 15_{i,j} of the insole 1 and that is used to determine the noise of the emitted data from the sensing units 15_{i,j} and a second threshold value that takes into account the variability of the sampled data d(i,j,t) by each sensing unit 15_{i,j} of the insole 1 and that is used to determine whether a possible fault is about to occur, as will be explained in the following.

The offload calibration step 51 comprises a first offload threshold calculation step 511 and a second offload threshold calculation step 512. The onload calibration step 53 comprises a first onload threshold calculation step 531 and a second onload threshold calculation step 532. The calibration process 50 further comprises a first threshold validation step 541 and a second threshold validation step 542 in which the calculated threshold values are validated for future use, as explained in the following. The calibration process 50 is based on the statistical distribution of the activation levels of the sensing units 10.

The offload calibration step 51 comprises a first offload threshold calculation step 511 in which a first offload threshold value TA1_{i,j} is calculated for each sensing unit 15_{i,j} and a second offload threshold calculation step 512 in which a second offload threshold value TA2_{i,j} is calculated for each sensing unit 15_{i,j}. The onload calibration step 53 comprises a first onload threshold calculation step 531 in which at least one or a plurality of first onload threshold value/values TC1_{i,j} is/are calculated for each sensing unit 15_{i,j} and a second onload threshold calculation step 532 in which a second threshold onload value TC2_{i,j} is calculated. The first offload threshold TA1_{i,j}, first onload threshold TC1_{i,j}, second offload threshold TA2_{i,j} and second onload threshold TC2_{i,j}, are calculated for each sensing unit 15_{i,j}. The calculation of the threshold is the same for each sensing units 15_{i,j} therefore in the following the first and second offload and onload threshold values will be indicated as TA1, TC1, TA2, TC2 respectively for clarity sake in the description.

The first and second offload threshold values TA1, TA2 measure the noise of the system, the first offload threshold value TA1 the basal noise connected with the amplitude of the sampled values and the second offload threshold value TA2 the noise connected with high frequency noise.

The first offload threshold value TA1 takes into account the amplitude distribution of the sampled data and it is calculated as a certain percentile of the distribution of the sampled values and it is used for determining if a value is to be considered as noise or as a real “measure”.

The second offload threshold value TA2 can be ascribed to a measure of variability between the sampled values over time and it is used by the fault check process 55 to determine whether a possible fault is about to occur. The first and the second threshold values TA1, TA2 are calculated a plurality of times in the offload calibration step 51, and then the different values are compared one another to evaluate the stability/variability of the threshold values obtained and thus the reliability of these values.

The first offload threshold value TA1 and second offload threshold value TA2 are calculated as explained in the following. The calibration process 50 is performed over the threshold calibration period TCP. The calibration period TCP is divided in a number of threshold recording periods nR each indicated as threshold recording period “Rp” with a length Rpl in the range of about 1-60 s, preferably 10 s. For each threshold recording period Rp a first offload threshold value TA1(Rpᵢ) and a second offload threshold value TA2(Rpᵢ) with i=1:nR are calculated, where Rpᵢ indicates the i^{th} (out of nR) recording period Rp of the calibration period TCP. For example, if the threshold calibration period TCP is 60 s and the length of the threshold recording period Rpl is 10 s and the sampling frequency Fs=1000 Hz the threshold calibration period TCP is divided in 6 threshold recording periods Rpᵢ (i=1:6), each composed of 10.000 data samples Rplₛ. In this example the first and second threshold offload value TA1(Rpᵢ) and TA2(Rpᵢ) are calculated 6 times in the calibration period TCP, a first and second offload threshold value for each threshold recording period Rpᵢ, and then the different threshold values obtained in each recording period are processed to be validated i.e. to determine the first and second threshold value for each sensing unit 15_{i,j} for the threshold calibration period TCP, as explained in the following.

In the following, first it will described the offload calibration step 51, i.e. it will be described how to calculate the first and second offload threshold value TA1(Rpᵢ) and TA2(Rpᵢ) for a threshold recording period Rpᵢ, then it will be described the threshold validation steps 541, 542 through which finally the first and second offload threshold values TA1 and TA2 are determined in the calibration process 50. In the validation process 54 the stability of the first and second offload threshold values of TA1(Rpᵢ) and TA2(Rpᵢ) over time and space (i.e., across the sensing unit layers) is evaluated, as explained in the following. As one possibility the first and second threshold offload values TA1 and TA2 may be calculated by averaging the respective values obtained in each threshold recording period “Rp” TA1(Rpᵢ) and TA2(Rpᵢ) across the number of threshold recording periods nR. In the offload calibration step 51 the sampled data d(i,j, Rpᵢ) of one threshold recording period Rpᵢ and from one sensing unit 15_{i,j} are divided into overlapping recording windows Rw (FIG. 11A).

The number of recording windows is nW, Rwᵢ indicates the i^{th} recording window out of nW.

Each recording window Rwᵢ of the recording windows Rw, where i=1:nW, has a window length Rwl between 0.01 and 1 s, preferably 0.1 s and in each recording window Rwᵢ a number of recording window samples Rwlₛ=Rwl*Fs is considered. The sampled data of one sensing unit 15_{i,j} and for the recording window Rwᵢ is indicated as d(i,j, Rwᵢ). Each two consecutive recording windows have a recording windows overlap Ro in which a number of overlap data samples Roₛ is provided. These data samples are shared between two consecutive recording windows Rwᵢ. For example, if the first recording window Rwᵢ has 10 samples, i.e. the data samples ranging from 1 to 10, and if the recording windows overlap Ro has 5 samples Roₛ, then the second recording window Rw₂ considers the data samples ranging from 5 to 15. The number of recording windows nW is calculated by the following formula:

nW=floor(Rplₛ−Roₛ/(Rwlₛ−Roₛ)) formula 1

where “floor” indicates the integer part of the number.

In the first offload threshold calculation step 511 for calculating the first offload threshold value for each recording period, TA1(Rpᵢ), the acquisition device 2 computes the maximum of the sampled data d(i,j,Rwi) in each recording window Rwᵢ thus obtaining a first threshold offload value of the period Ta1(Rwᵢ), where Ta1(Rwᵢ)=Max(d(i,j, Rwᵢ)). The first threshold offload value of the different recording windows Ta1(Rwᵢ) are then stored in the memory unit 24. At the end of the process a number nW of first threshold offload value Ta1(Rwᵢ) values is stored in the memory unit 24.

Then the acquisition device 2 calculates a certain percentile threshold of the first offload threshold value of the recording window Ta1(Rwᵢ), usually 95-99%, and adds a safety margin thus obtaining the first threshold offload value TA1(Rpᵢ) for each sensing unit 15_{i,j} and each threshold recording period Rpᵢ. The value of the safety margin may be chosen during manufacturing of the system of the invention. Therefore, the first offload threshold value of the recording period TA1(Rpᵢ) corresponds to the 95%-99% percentile of the distribution of the sampled data, plus a safety margin. The first threshold offload value of each recording period TA1(Rpᵢ) roughly corresponds, for each sensing unit 15_{i,j}, more or less to the “minimum value” sampled by the sensing unit 15_{i,j} during the threshold recording period Rp, at the registration condition above which the sensing unit 15_{i,j} may be considered active.

The second threshold offload value of the recording period TA2(Rpᵢ) takes into account the variability over time of the sampled data d(i,j, Rpᵢ) by each sensing unit 15_{i,j} and it is calculated in the following way.

In the second offload threshold calculation step 512, the acquisition device computes the second offload threshold value Ta2(Rwᵢ) in each recording window Rwᵢ where i=1:nW. If the number of data samples within each recording window is Rwlₛ then Ta2(Rwᵢ) is calculated according to the formula below:

Ta2(Rwᵢ)=Id(tf)−d(tf−1)I+Id(tf−1)−d(tf−2)I+ . . . Id(2)−d(1)I formula 2

where d(tf) is the last sample of the data d(i,j, Rwᵢ) relative to the recording window Rwᵢ and sensing unit 15_{i,j}.

In other version of the method the following general formula may be used:

Ta2(Rwᵢ)=k0ld(tf)−d(tf−1)I\hat{ }n+k1Id(tf−1)−d(tf−2)|\hat{ }n+ . . . kpld(2)−d(1)|\hat{ }n formula 3

where k0, k1, . . . kp are multiplicative factors for weighting the various addends, for example the factors may have an exponential decrease, n is a number, usually 1 and varied based on the importance which should be given to the outliers. Different values of n can be used within formula 3 for each addend.

After performing the calculation of second offload threshold value Ta2(Rwᵢ) for each recording window, a number nW of second offload threshold value Ta2(Rwᵢ) is available and stored in the memory unit 24.

In the same way, as for the first offload threshold value TA1, a value is calculated over the second offload threshold value of each recording window Ta2(Rwᵢ) distribution according to a certain percentile (e.g. 99%) and this threshold is indicated as second threshold offload value TA2 for sensing unit 15_{i,j} and threshold recording period Rp.

The formula 2 and formula 3 above compare the sampled values obtained for each sensing unit 15_{i,j} in consecutive data samples within a recording window Rwᵢ, therefore the greater the difference between two consecutive values the greater the calculated second offload threshold value Ta2(Rwᵢ).

The validation process 54 is then executed for validating the first and second offload threshold values. The validation process 54 comprises a first threshold validation step 541 for checking if the calculated first and second offload threshold values TA1(Rpᵢ), TA2(Rpᵢ), calculated for the various threshold recording periods (Rpᵢ) are stable over time (i.e., over the threshold calibration period TCP, FIG. 10), and a second threshold validation step 542 for checking if the calculated first and second offload threshold values TA1, TA2 are reliable, i.e. stable over the sensing units 15_{i,j}. In the first threshold validation step 541, the first and second threshold offload values TA1(Rpᵢ), TA2(Rpᵢ) from a sensing unit 15_{i,j} in the different threshold recording periods (Rpᵢ) are considered to be validated if the values are within a certain variability range over time. In the second threshold validation step 542, the first and second threshold offload values TA1(Rpᵢ), TA2(Rpᵢ) from a sensing unit 15_{i,j} are considered validated if their value is close to the corresponding values obtained from neighboring sensing units.

In the first threshold validation step 541 it is provided for calculating multiple times over time the first offload threshold value TA1(Rpᵢ) for each sensing unit 15_{i,j} a plurality of first offload threshold value TA1(Rpᵢ) is calculated one of each threshold recording periods Rpᵢ, in which the threshold calibration period TCP is divided in the manner discussed above. Therefore, a plurality of first threshold offload values i.e., TA1(Rp₀), TA1(Rpᵢ), . . . TA1(Rp_{nR}) is calculated for different threshold recording periods Rpᵢ over the threshold calibration period TCP.

The different first threshold offload values TA1(Rp₀), TA1(Rp₁), . . . TA1(Rp_{nR}) calculated over the different threshold recording periods Rpᵢ are then compared one with another in the computing device 4 to measure the variability of the values obtained in the different threshold recording periods Rpᵢ, i.e. over time. If, for each sensing unit 15_{i,j}, the first threshold offload values calculated over the different threshold recording periods Rpᵢ, TA1(Rp₀), TA1(Rp₁), . . . TA1(Rp_{nR}) are within a certain variability range S, then the calculated first threshold offload value is considered stable over time.

Only if the threshold value is considered stable over time then, a mean value TA1 of the calculated first threshold offload values TA1(Rp₀), TA1(Rp₁), . . . TA1(Rp_{nR}) is calculated, and stored in the memory unit 24 (See FIG. 10) as the first offload threshold value TA1. Any one of the several kinds of means, for example the arithmetic mean can be used for calculating the mean value of the calculated first offload threshold values TA1 for each sensing unit 15_{i,j}. The mean value obtained is considered to be the first offload threshold value TA1 for each sensing unit 15_{i,j}.

The first offload threshold value TA1 will indicate the “zero” condition for the analysis system 100.

In other words, any value of sampled data d(i,j,t) which is lower than the first threshold offload value TA1 will be considered as “0” and thus discarded, as explained in the following. Any value of sampled data d(i,j,t) which is lower than first offload threshold value TA1 will be considered as “noise” thus discarded as not corresponding to a real modification of the emitted physical condition. If for some of the sensing units 15_{i,j} the calculated first threshold offload values TA1(Rp₀), TA1(Rp₁), . . . TA1(Rp_{nR}) in the recording periods Rpi are outside the preset variability range S, then the calculated first threshold offload values are discarded and the corresponding sensing unit(s) 15_{i,j} is/are not considered in the following operating process 60 or in general in future evaluations.

The variability range S depends on the precision required for the analysis system 100, usually about 5-40% of the value of the mean, preferably within 30% of the mean, more preferably the variability range S is 10% of variability.

In some cases the first threshold offload values outside the variability range S may still be stored in the memory unit 24. The first threshold validation step 541, is repeated also for the second offload threshold values TA2 and will not be repeated in the following. A mean value of the second offload threshold values of the different recording periods TA2(Rpᵢ), is then stored in the memory unit 24 and indicated as second offload threshold value TA2.

In the first threshold validation step 541, a different value of the variability range for the first and the second offload values TA1 and TA2 may be set.

Then, the second threshold validation step 542 is executed for checking the reliability of the calculated first and second offload threshold values TA1, TA2, i.e. the stability of the calculated first and second offload threshold values TA1, TA2 from the different sensing units 15_{i,j} over space. In the second threshold validation step 542 it is provided for checking if the first and second offload threshold values TA1 and TA2 obtained by the first threshold validation step 541 for each sensing unit 15_{i,j} from neighboring sensing unit 15_{i,j} are within a predefined further variability range S′. The first and second threshold offload values TA1 and TA2 are compared across neighboring sensing units and checked to be in a predefined further variability range S′, i.e. it is checked if threshold values from neighboring sensing units are close one to another or if there is a high difference between values from neighboring sensing units. It is considered that neighboring sensing units should give values close one to another, as the degree of activation of neighboring sensing units must be similar.

If this does not happen and, on the contrary, values from neighboring sensing units differ more than the predefined further variability range S′, there is a problem in at least one of the values, i.e. in at least one sensing unit. This could be caused for example from an unexpected noise or from a faulty sensing unit 15_{i,j}. In this case, the corresponding first and second offload threshold values TA1 and TA2 cannot be taken into account, therefore these values are not stored in the memory unit 24 and are not used in the operating process 60, as explained below.

The first and second offload threshold values TA1 and TA2, obtained by the different sensing units 15_{i,j} and which have not been discarded in the first threshold validation step 541 are compared one with another across neighboring sensing units and checked to be within a predefined further variability range S′ that is memorized in the memory unit 24 of the acquisition device 2.

For each sensing unit, the comparison is made with sensing units in the corresponding positions but from neighboring different layers and with sensing units of the same layer but from different neighboring positions.

For example, the first offload threshold value TA1_{i,j} obtained for the sensing unit 15_{i,j} in the first threshold validation step 541 is compared with the first threshold value obtained from the corresponding sensing unit in an adjacent layer, e.g. from the sensing units 15_{i-1,j} and 15_{i+1,j} and it is also compared with the first threshold values obtained from neighboring sensing units of the same layer i.e. 15_{i,j-1}, 15_{i,j+1}. If the first offload threshold value TA1_{i,j} obtained from neighboring sensing units are within the predefined further variability range S′, they are considered validated and can be stored in the memory unit 24. The same procedure is repeated for the second offload threshold values TA2_{i,j}.

The offload thresholds values TA1_{i,j} and TA2_{i,j} calculated as discussed above that have passed both the first and the second threshold validation steps 541 and 542 discussed above are written into the memory unit 24 of the acquisition device 2 for further use, a mean value of the offload thresholds values TA1_{i,j} and TA2_{i,j} is stored for each sensing unit 15_{i,j}, i.e. the offload thresholds values obtained in the first threshold validation step 541. As indicated above, any possible mean can be used, for example the arithmetic mean or the median mean. In this way, for each sensing unit 15_{i,j} only the offload thresholds values TA1_{i,j} and TA2_{i,j} which are stable over time and which are coherent with the offload thresholds values TA1_{i,j} and TA2_{i,j} from neighboring sensing units are taken into account and a “mean” value of these useful offload thresholds values TA1_{i,j} and TA2_{i,j} is stored in the memory unit 24. The further variability range S′ is preferably within 15% of the mean.

This implies that “wrong” values are not stored and, therefore, it is avoided that the wrong values affect the future analysis. It could happen that offload thresholds values TA1_{i,j}, TA2_{i,j} for only some of the sensing units 15_{i,j} of the insole 1 pass the validation process 54 and are then stored in the memory unit 24.

The offload threshold values TA1_{i,j} and TA2_{i,j} which don't pass the validation process discussed above are discarded and the corresponding sensing unit 15_{i,j} is(are) considered to be faulty and the user is advised to check the setup, replace them. The calibration process may be repeated by the user multiple times before deciding to replace the system or the sensing units. The user receives a signal containing the information about the faulty sensing unit 15_{i,j}.

In a version of the method it could be provided for storing in the memory unit and/or using also the offload threshold values TA1_{i,j}, TA2_{i,j} coming from the sensing units 15_{i,j} for which the calculated values have not been validated, in this case the user is nonetheless informed about the possible problematicness of the corresponding sensing unit(s). Basing on this warning the user can decide if keeping to consider the problematic sensing unit(s) 15_{i,j} or to disregard it (them). Additionally, the user can also decide to substitute one or more insole layers 12ᵢ of the insole 1, if for example in the same layer there are many faulty or problematic sensing units 15_{i,j}, or also to substitute the insole 1 if the number of the faulty or problematic sensing units 15_{i,j} is too high.

Depending on the task to be subsequently performed, it could be decided if the faulty sensing units 15_{i,j} are to be kept or disregarded. If, in particular, none of the first and second threshold validation steps 541, 542 discussed above is successful for each sensing unit 15_{i,j} it is highly probable that the environment is too noisy and that this noise changes abruptly over time, or that the whole insole layer 12ᵢ needs to be replaced or ignored in future evaluations.

Due to the number of layers of the insole 1, also disregarding one of the problematic layers reliable evaluations can be performed. Then the method provides for performing the onload calibration step 53, in which the user wearing the insole 1 stays still at first and then performs several repetitions of the tasks he would like to accomplish (e.g., climbing stairs, walking, standing, sitting) for a further threshold calibration period lasting a certain amount of time of about 1-2 minutes and indicated further threshold calibration period TCP′.

The onload calibration step 53 comprises a first onload threshold calculation step 531 to calculate the first onload threshold value TC1_{i,j,e} for each sensing unit 15_{i,j} and with a certain level of activation Iₑ, where e=1:E of the plurality of levels of activation E and a second onload threshold calculation step 532 to calculate the second onload threshold value TC2_{i,j} for each sensing unit 15_{i,j} as will be explained in the following. The calculation of TC1_{i,j},e is similar to the calculation of the first threshold offload value TA1_{i,j} with the differences explained hereafter.

It should be noted that while the procedure to calculate the onload threshold TC1_{i,j,e} is similar to the procedure used to calculate the first threshold offload value TA1_{i,j} the length of the further threshold calibration period TCP′ and the length of the further recording windows Rwi′ etc. can be different than those used for the for the first threshold offload value TA1 even if their role remains the same. The further threshold calibration period TCP′ for the onload threshold TC1_{i,j,e} is to be chosen in a way that assures a certain number of repetitions of the same movements by the user. During the further threshold calibration period TCP′, the sensing units 15_{i,j} sense the pressure exerted by the user and send the sampled data d(i,j,t) through the acquisition device 2 to the computing device 4.

The further threshold calibration period TCP′ having a further threshold calibration period length TCPI′ about 0-300 s, preferably 120 s, is divided in a number of threshold recording periods nR indicated as onload threshold recording period Rp′ with length of the onload threshold recording period Rpl′, in the same way as explained before for the offload first and second threshold values TA1 and TA2.

The sampled data d(i,j,t) are transformed using the method and the formulas seen above for the calculation of the first and second offload threshold values TA1 and TA2 respectively and will not be repeated.

As explained below, for each sensing unit 15_{i,j} only the sampled data d(i,j,t) that, after being transformed using the formulas and method seen for the calculation of the first and second offload threshold values TA1 and TA2, are higher than the first offload threshold value TA1 obtained at the validation step 54, (i.e. the values which can be considered above the noise level of the analysis method 200) and lower than the second threshold offload value TA2 obtained at the validation process 54 (i.e., the values which are considered too noisy) are considered for the calculation of the first and second onload threshold values TC1_{i,j,e} and TC2_{i,j}. In other words, for each one of the sensing units 15_{i,j} for which the sampled data d(i,j,t) passed the validation step 54, a plurality of first onload threshold value TC1_{i,j,e} corresponding to predetermined degrees of activation of the sensing unit 15_{i,j} are calculated. According to the analysis method of the invention, for each sensing unit 15_{i,j} there are calculated many different first onload threshold values TC1_{i,j,e}, e=1, . . . E, each corresponding to a different degree of activation of each sensing unit 15_{i,j} i.e. to different percentile of activation for each sensing unit, for example 30%, 50%, 80% activation of the sensing unit 15_{i,j}.

Since for obtaining the first onload threshold values TC1_{i,j,e}, e=1, . . . E the same calculation will be performed for all the sensing units 15_{i,j} for which the sampled data d(i,j,t) are higher than the first offload threshold value TA1_{i,j}, for clarity sake these values will be indicated as first onload threshold values TC1ₑ.

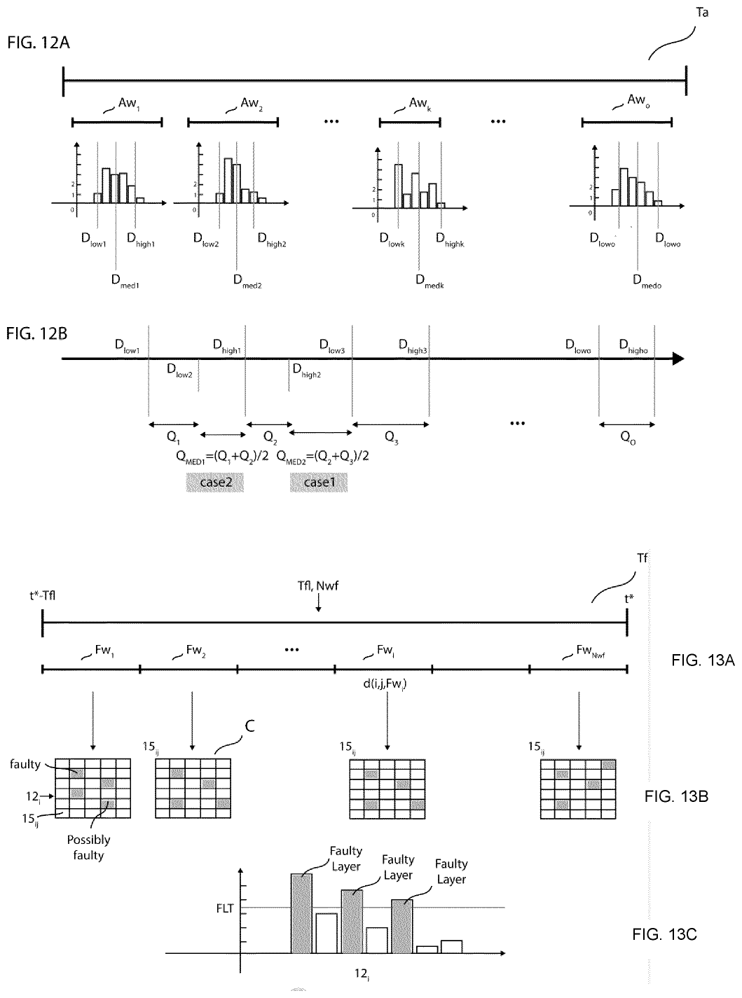

For sake of clarity while the first offload threshold value of the recording period TA1(Rpi′), as shown in FIG. 12, is computed as a certain percentile (e.g., 95%) of the distribution of the first offload threshold value TA1(Rwi), here several first onload threshold values TC1ₑ for each recording period are computed as different percentiles (e.g., 30%, 50%, 80%) of the corresponding distribution of first onload threshold values of each recording window TC1ₑ(Rwi′). In this way, in each onload threshold recording period Rpᵢ′ it is obtained a plurality of first onload threshold values TC1e each first onload threshold value corresponding to a different level of activation le of the sensing units.

Considering all the onload threshold recording periods Rpᵢ′ (see FIG. 11) of the further threshold calibration period TCP′, there are obtained several values of the first onload threshold values of each recording period TC1ₑ(Rpi′) (see FIG. 10) with e=1, 2, . . . , E each corresponding to a different percentile of the activation.

For obtaining the first onload threshold values TC1e, the first onload threshold values TC1e(Rpi′) of each threshold recording period Rpᵢ′ are averaged in one of the ways discussed above for the first offload threshold value TA1.

There is therefore obtained for each sensing unit 15_{i,j} a plurality of first onload threshold values TC1e(Rpi′) for each percentile of activation each one of which corresponding to a mean value of the first onload threshold values TC1e(Rpi′) obtained in the different threshold recording periods Rpi. Since sampled data d(i,j,t) that are below the first offload threshold value TA1 are discarded, then all the first onload threshold values TC1e(Rpi′) will be higher than the first offload threshold value TA1_{i,j}.

For each sensing unit 15_{i,j} each one of the first onload threshold values of each recording period TC1e (Rpi′) corresponds to the predefined percentile “e” of the distribution corresponding to a different level of activation le of the sensing units 15_{i,j}. For each sensing unit 15_{i,j} there are defined many levels of activation and for each one of them a corresponding first onload threshold value is calculated: a lowest first onload threshold value TC1₁ corresponding to the lowest level of activation I₁, a second first onload threshold value TC1₂ corresponding to the second level of activation I₂, etc. Correspondingly, for each sensing unit 15_{i,j} many intervals of activation le where e=1, . . . E are defined: a first interval of activation from zero up to the lowest first onload threshold value TC1₁, a second level of activation between the lowest onload threshold value TC1₁ and the second first onload threshold value TC1₂, etc.

In the first onload threshold calculation step 531 many different first onload threshold values TC1ₑ in each onload threshold recording period Rpi′ for different levels of activation are calculated: increasing the number of first onload threshold values the number of the intervals of the sampled data to be calculated and analyzed is increased, and correspondingly it is increased the precision of the system, as explained better in the following. The number of the different levels of activation may be chosen by the user, usually about 3-30, preferably about 4-10 first onload threshold values TC1ₑ are calculated.

According to the invention it is provided for calculating at least one first onload threshold value corresponding to a preset level of activation of the sensing unit 15_{i,j}.

As indicated above, the first onload threshold calculation step 531 is performed only for those sensing units which have not been discarded in the validation process 54 when validating the first and the second offload threshold values TA1, TA2. For example, there are defined for each sensing unit 15_{i,j} four levels of activation corresponding respectively to 25%, 50%, 75% and 100%. For each level of activation le a corresponding first onload threshold value is calculated, therefore it is provided for calculating four first onload threshold values TC1_{i,j,1}, TC1_{i,j,2}, TC1_{i,j,3} TC1_{i,j,4}.

A comparison is made between the sampled data and the first onload threshold value in the evaluation phase 61, in particular in the first threshold comparing process 50′A in which, in the onload condition, the sampled data are compared with the first onload threshold values TC1_{i,j,e} previously calculated and the level of activation le of the sensing units 15_{i,j} is calculated. For example there are defined four intervals of activation I₁₋₄: the first interval of activation I₁ for a degree of activation comprised between the first offload threshold value TA1_{i,j} and ≤25%, second interval of activation I₂ in which the degree of activation is >25% and ≤50%, etc. Indicating as “A” the value emitted by one sensing unit 15_{i,j} at a time t_{A} the following relationships are used for evaluating the sampled data d(i,j,t_{A}):

If TA1_{i,j}<A≤TC1_{i,j,1}=>I₁, then the degree of activation is lower than or equal to the lowest first threshold value TC1_{i,j},1 and it is considered that the sensing unit 15_{i,j} has a I₁ level of activation;

If TC1_{i,j,1}<A≤TC1_{i,j,2}=>I₂, then the degree of activation is lower than or equal to TC1_{i,j,2} but greater than TC1_{i,j,1} and it is considered that the sensing unit 15_{i,j} has a I₂ level of activation;

If TC1_{i,j,2}<A≤TC1_{i,j,3}=>I₃, then the degree of activation is lower than or equal to TC1_{i,j,3} but greater than TC1_{i,j,2} and it is considered that the sensing unit 15_{i,j} has a I₃ level of activation;

If TC1_{i,j,3}<A≤TC1_{i,j,4}=>I₄, then the degree of activation is lower than or equal to TC1_{i,j,4} but greater than TC1_{i,j,3} and it is considered that the sensing unit 15_{i,j} has a I₄ level of activation.

If the level of activation of the sensing unit 15_{i,j} is so low that the sampled value is lower than the corresponding first threshold value TA1_{i,j}, that sensing unit is considered not active, and the level of activation is set to 0.

The above indicated calculation is repeated for each sensing unit 15_{i,j} which have not been discarded in the validation process 54 and on the sampled data d(i,j,t) produced in the further threshold calibration period TCP′. In the first threshold comparing process 50′A the sampled data d(i,j,t) are compared with the subsequent first onload thresholds values TC1_{i,j,1}, TC1_{i,j,2} . . . until it is reached the first onload threshold value TC1_{i,j,e} that is greater than or equal to the sampled data and then the sampled data is labelled with the corresponding interval Iₑ.

The provision of many different first onload thresholds values TC1_{i,j,2}, TC1_{i,j,2} . . . allows to be obtained an information about the level of activation of the insole 1 which is independent from the position and specific characteristics of the sensing unit.

Based on the results of the first threshold comparing process 50′A, for each sensing unit 15_{i,j} the sampled data d(i,j,t) is labelled in a manner corresponding to the interval of activation I₁₋₄ and sent to the operating process 60 as calibrated data dc(i,j,t) from where they are stored in the memory unit 24 and/or sent to the computing device 4, as explained below. As indicated above, the number of the intervals of the activation level may be chosen by the user: by increasing the number of the intervals the precision of the system and of the method is increased.

The level of activation corresponding to the different first onload threshold values TC1_{i,j,e} may be chosen by the user based on the task to be performed, for example walking, running . . . , etc. Also, the number of levels of activation E in which the overall range is divided is also chosen by the user.

The provision of many different first onload threshold values allows to take into account the different activation phases and the different levels of activation of the different sensing units 15_{i,j} of the insole 1 during the task. Furthermore, the provision of different first onload thresholds values allows a more reliable system to be obtained. Additionally, it also allows to obtain a simpler and faster analysis method which is at the same time reliable, as explained below. This allows the data stored and/or transmitted to be reduced, reducing the quantity of data exchanged or stored in the memory unit 24. The provision of many different first onload threshold values allows the amplitude of the signal from the sensing units to be quantified and to divide the signal from the sensing units into discrete intervals. This also allows to take into consideration only the differences in the signal which are considered useful for the real situations.

Changing the number of the levels of activation i.e. the number of first onload threshold value and, consequently, the number of intervals, it is possible to adjust the sensitivity of the method of the invention, i.e. to adjust the variation in the signal from the sensing units which are deemed necessary for causing a change in the interval of activation. This also allows to define the sensitivity of the system and of the method of the invention, i.e. the variations in the signal which are reflected in a difference in the data stored in the memory unit 24 or sent to the computing device 4.

By increasing the number of the first onload threshold values TC1_{i,j,e} the sensitivity of the method is increased, i.e. minor differences in the signal are taken into account as they correspond to a different level of activation, decreasing the number of different first onload threshold values the sensitivity is decreased, i.e. minor differences in the signal are not taken into account as they are considered to correspond to the same level of activation. In any case, minimal variations in the signal are not taken into account, they are deemed connected with the same level of activation of the sensing unit. Therefore, it is possible to properly scale the signal from the sensing unit.

It is also possible to adjust the variation in the original physical values that produce a different final signal, i.e. the differences that should be taken into account by the method.

The first onload threshold values are calculated only for those sensing units 15_{i,j} which have passed the validation process, i.e. those sensing units 15_{i,j} for which offload threshold values have been stored in the memory unit 24 and the calibration process. In another version of the analysis method it is provided for considering in the onload calibration step 53 also the sensing units 15_{i,j} for which the sampled value is lower than or equal to the first offload threshold value (≤TA1). In this case the degree of activation of the first interval of activation I₁ is set to “0”.

It is possible to calculate different sets TC1E of first onload threshold values TC1_{i,j,e} each set corresponding to a different activity or a mix of these, like for example walking, running, obstacle racing, etc., the different sets TC1E of first onload threshold values having also a different number of elements, i.e. a different number of first onload threshold values TC1_{i,j,e}. Having different sets of first onload threshold values TC1E allows for example to compensate the changes in the sampled data effected e.g. by the tightness of the shoes.

The different sets TC1E of first onload threshold values TC1_{i,j,e} are stored in the memory unit 24 and, basing on the real condition of use, the correct set TC1E of first onload threshold value TC1_{i,j,e} is used.

In the second onload threshold calculation step 532, the second onload threshold value TC2 is calculated in a manner similar to that described with reference to the second offload threshold value TA2 and will not be repeated in the following. The calculated first and second onload threshold values TC1_{i,j,e}, TC2_{i,j} obtained by the first and second onload threshold calculation step 531, 532 respectively are stored in the memory unit 24 of the acquisition device 2 for further use.

Also in this case, a mean value of each one of the first and second onload threshold values TC1_{i,j,e}, TC2_{i,j} is stored in the memory unit 24. The calculated first and second onload threshold values TC1_{i,j,e}, TC2_{i,j} obtained by the onload calibration step 53 are then subjected to the validation process 54 so that the first and second onload threshold values TC1_{i,j,e}, TC2_{i,j} are validated, the validation process 54 is the same discussed above for the offload first and second threshold values TA1, TA2 and will not be discussed in the following.

At the end of the calibration process 50 the threshold values TA1_{i,j}, TA2_{i,j}, TC1_{i,j}, TC2_{i,j} calculated as described above for the desired condition of use and validated are stored in the memory unit 24 of the acquisition device 2 and sent to the evaluation phase 61. At the end of the calibration process 50 the level of activation le are also stored in the memory unit 24 of the acquisition device 2 and sent to the evaluation phase 61. With the calibration process 50, the noise of the emitted data in baseline condition is quantified, which depends on the environments and setup. The calibration process 50 can be made by the user before using the analysis system 100 storing the threshold values in the memory unit 24. Alternatively, the calibration process 50 can be made by the producer of the insole and the user obtain the analysis system 100 with already stored threshold values for the different conditions of use.

The sampled data d(i,j,t) are compared in the evaluation phase 61 with the threshold values stored in the memory unit 24, if the analysis system 100 is in the offload condition the sampled data d(i,j,t) are compared with the offload threshold values TA1_{i,j} and TA2_{i,j}, if the analysis system 100 is in the onload condition, the sampled data d(i,j,t) are compared with the onload threshold values TC1_{i,j,1} TC1_{i,j,2} etc. and TC2_{i,j}, as explained in the following.

In the first threshold comparing process 50′A the current sampled value collected at time t* and indicated as d(i,j,t*) is compared with the first offload and onload threshold values TA1_{i,j} and TC1_{i,j,e} as explained in the following in the first threshold comparing process 50′A. Nevertheless for the comparison of the current sampled value d(i,j,t*) with the second offload and onload threshold values, in the second threshold comparing process 50′B, the sampled data d(i,j,t) are transformed using the formulas and procedures discussed above for calculating the second offload and onload threshold values TA2_{i,j} and TC2_{i,j} and then compared with the second threshold values, as explained below. For example, to compare the current sampled value d(i,j,t*) at time t* with the second offload threshold value TA2_{i,j}, it is necessary to consider an interval of data having the same length Rwl (in seconds) or Rwls (in samples) of the calibration window Rw on which the second offload threshold values TA2_{i,j} has been calculated, that is d(i,j,[t*−Rwl, t*]). The sampled data d(i,j,[t*−Rwl, t*]) i.e. the data ranging from the current time t*t time t*−Rwl in the past is transformed to obtain transformed data d°(i,j,t*) using the following formula:

d°(i,j,t*)=T(d(i,j,[t*−Rwl,t*])) formula 4

where T indicates that the transformed data d°(i,j,t*) are obtained using the formula 2 or 3.

The sampled data d(i,j,[t*−Rwl, t*]) are thus transformed using the formula 1 obtaining transformed data d°(i,j,t*) and the transformed data d°(l,j,t*) are then compared with the second offload and onload thresholds values TA2_{i,j} and TC2_{i,j}.

This comparison is performed in the second threshold comparing process 50′B, depending on the condition of use. For example, as soon as the user turns the system on (while not wearing it), the second offload threshold value TA2_{i,j} is used. If the user wears the device, the second onload threshold value TC2_{i,j} is used. During the second threshold comparing process 50′B, if the transformed data d°(i,j,t*) are higher than the second threshold offload value TA2_{i,j} for the offload condition, or are higher than the second threshold onload value TC2_{i,j} for the onload condition respectively, for more than a certain number of times nF within a Fault Check Time Tf (preferably the same as the one used in the fault check process 55—see FIG. 13), then the relative sensing unit 15_{i,j} is deemed faulty and not used in the following phases. In the first threshold comparing process 50′A if at the current time t*, the current sampled value d(i,j,t*) from sensing unit 15_{i,j}, does not exceed the first offload threshold value TA1 of that sensing unit 15_{i,j}, i.e. d(i,j, t*)≤TA1(i,j) then the current sampled value d(i,j,t*) is not transmitted to the computing device 4 and it is not stored in the memory unit 24 thus saving energy and memory. This value is considered below or at the noise level of that sensing unit 15_{i,j}. Thus the corresponding sensing unit 15_{i,j} is considered not active. Therefore if provided, the output interface TTL trigger is left to 0 (off) and the data are not transmitted to the computing device 4.

It should be noted that the current sampled value d(i,j,t*) at time t* changes for each sensing unit 15_{i,j} and over time since during the performing of the task by the user the level of activation of the different sensing units 15_{i,j} changes. In the onload condition, if at the time t*, the current sampled value d(i,j,t*) exceeds the first offload threshold value TA1, i.e. d(i,j,t*)>TA1 then the current sampled value d(i,j,t*) is compared with the first onload threshold values TC1e and a signal corresponding to the level of activation, le, of the sensing unit 15_{i,j}, is transmitted to the computing device 4 and/or stored in the memory unit 24. If at the time t*, the current sampled value d(i,j,t*) is lower than or equal to the first onload threshold value TC1, d(i,j,t*) TC1 the value is discarded and, if provided, the TTL trigger is set to 0. If at the time t*, a current sampled value d*(i,j, t*) is greater than first onload threshold value TC1, d(i,j,t*)>TC1 the current sampled value is kept and memorized in the memory unit 24 and/or sent to the computing device 4, if provided the TTL trigger is set to 1.

In case a plurality of first onload threshold values TC1₁, TC1₂ . . . are provided, each one of which corresponding to a different degree of activation of the sensing units, the comparison is made with each first onload threshold values. If at the time t*, the current sampled value d(i,j,t*) is lower than or equal to the lowest onload threshold value TC1ᵢ, d(i,j,t*)≤TC1₁ the value is discarded and not sent to the computing device or stored in the memory unit 24. Plus, if provided, the TTL trigger is set to 0. In this case it is in fact considered that the sensing unit 15_{i,j} is not active at the time t*. If at the time t*, the current sampled value d(i,j,t*) is higher than the lowest first onload threshold value TC1₁, the value is compared with the subsequent first onload threshold values TC1ₑ until it is found a first threshold value that is greater than or equal to the current sampled value d(i,j,t*).

Then the current sampled value d(i,j,t*) is labelled with the level of activation Iₑ for which the following relationship is satisfied: TC1ₑ<d(i,j,t*)≤TC1ₑ₊₁, and stored in the memory unit 24 and/or sent to the computing device 4. If a plurality of first onload threshold values TC1ₑ, are available in the memory unit 24 of the acquisition device 2 and if deemed useful by the user, the current sampled value d(i,j,t*) is compared with any of the first onload threshold values TC1ₑ stored in the memory unit 24 and the current sampled value d(i,j,t*) lower than or equal to a specific first onload threshold values TC1ₑ thus falling within a specific threshold interval is labelled with the corresponding level of activation le and stored in the memory unit 24 and/or sent to the computing device. For a certain range of variation of the signal from the sensing units the same information is provided, i.e. the same interval is associated to a range of degree of activation of the sensing units.

Modifying the number of intervals Iₑ of the degree of activation, the extension range of the degree of activation is modified and, therefore, the sensitivity of the analysis method is adjusted. The current sampled values d(i,j,t*) that are greater than the lowest first offload threshold values TA1, d(i,j,t*)>TA1 are sent to the computing device 4 and/or stored in the memory unit 24 together with the code identifying the specific interval Iₑ. This comparison is repeated for all the sensing units 15_{i,j} of the insole 1, at the sampling frequency Fs within the first threshold comparing process 50′A. Only sampled data greater than the first offload threshold value TA1 are sent on to the other processes, for example the fault check process 55, the operating process 60, the gait segmentation process 80, as explained below. Therefore, the quantity of data stored and/or sent is greatly reduced. As indicated above, second threshold values are used to measure the noise, or the errors, on the measured value due for example to a faulty sensing unit 15_{i,j}, if a measured value is greater than the corresponding second threshold value it is likely that a problem exists.

The transformed data d°(i,j,t*)=T(d(i,j,[t*−Rwl, t*])), obtained as indicated above with formula 4, are thus compared with the second threshold value corresponding to the real condition of use, offload or onload. If the transformed data d°(i,j,t*) of a sensing unit 15_{i,j} is greater than the corresponding second threshold value TA2, or TC2 a corresponding warning signal is sent to the fault check process 55 if it exists or directly to the operating process 60 and stored in the memory unit 24. The current sampled value d(i,j,t*) which has overcome the evaluation phase 61, i.e. the first and second threshold comparing process 50′A, 50′B will be indicated as current calibrated value dc(i,j,t*) and is coded to TTL ports, serial or other equally-fast external communication interfaces and it is made available for future use to the operating process 60. Therefore, only a current calibrated value dc(i,j,t*) i.e. data produced by active sensing units 15_{i,j}, is sent to the operating process 60 and then to the computing device 4 and/or stored in the memory unit 24 by means of the memorization phase 62.

In other words, for the active sensing units 15_{i,j} at the current moment of time t* it is sent and/or stored a value which takes into account the single sensing unit and the current level of activation thereof.

The calibrated data dc(i,j,t) explains the activation level and the number of possible values that can depend on the number of onload thresholds used. For example, if only offload threshold TA1 is used, current calibrated value dc(i,j,t*) will be equal to “0” if d(i,j,t*)≤TA1 or a current calibrated value dc(i,j,t*) will be equal to “1” if d(i,j,t*)>TA1. As another example, if two first thresholds onload values TC1_{i,j,1} and TC1_{i,j,2} are used then the following relationships will be used:

| | |
|---|---|
| i.dc(i, j, t*) = 0 | if d(i, j, t*) ≤ TA1, |
| dc(i, j, t*) = I₁ | if TA1 < d(i, j, t*) ≤ TC1_{i, j, 1,} |
| dc(i, j, t*) = I₂ | if TC1_{i, j, 1} < d(i, j, t*) ≤ TC1_{i, j, 2} |
| dc(i, j, t*) = I₃ | if TC1_{i, j, 2} < d(i, j, t*). |

Therefore, with the first and second threshold comparing process 50′A, 50′B, at each current time t*, only data from some of the sensing units 15_{i,j} of the insole 1 are sent to the computing device 4 and/or stored in the memory unit 24 and possibly displayed on the display unit 6 of the computing device 4. This decreases the quantity of data transmitted from the acquisition device 2 to the computing device 4 and/or stored in the memory unit 24 increasing the efficiency of communication therebetween thus saving energy and memory. At the same time, it is assured that useful data are transmitted and used. Additionally, the level of analysis required by the computing device 4 is drastically reduced and the response time is consequently highly decreased.

Additionally, comparing the sampled data d(i,j,t) with the stored offload and onload threshold values TA, TC the influence of noise of the current sampled value d(i,j,t*) at current time t* is decreased and also the interference due to the normal use of the insole, movement, etc. are decreased. These advantages are obtained assuring at the same time the required reactiveness of the system.

The evaluation process 61 is repeated throughout the duration of the task accomplished by the user preferably at the sampling frequency Fs. However, in a further version of the method 200 the comparison of the evaluation process 61 is effected at a comparison frequency Fm lower than the sampling Frequency Fs, usually comprised between 1/10 and 1/100 of the sampling frequency Fs. In this case the current calibrated value dc(i,j,t*) obtained by the evaluation process 61 are obtained at a lower comparison frequency Fm than the sampling frequency Fs.

Thus, the method of the invention allows reliable data to be obtained and to obtain data with a very short delay, the delay is lower or equal to the comparison time Tm calculated as the inverse of the comparison frequency Fm. In summary, the first (taking into account of the amplitude of the signal) and the second (taking into account of the frequency of the signal) threshold values corresponding to the conditions of use, (offload, onload), are stored in the memory unit 24, either being calculated or provided ab initio from the producer of the insole 1. For the onload situation many first threshold values TCe are stored in the memory unit, each corresponding to a different degree of activation of the sensing unit.

During use, the sampled data d(i,j,t) are compared in the evaluation process 61 with the threshold values TA1, TA2, TC1e, TC2 corresponding to the condition of use to give a warning of possible breakages or faulty situation. The use of the first and second threshold values TA1, TA2, TC1e, TC2 gives inter alia the advantage of speed as it allows for example to activate on-off triggers quickly in 1-2 ms and to disregard if necessary some sensing units 15_{i,j}, for example those for which the values are lower than some threshold values or which are outside a certain variability range or which are faulty.

The comparison is done for each sensing unit 15_{i,j}, therefore it is obtained a map of the values from all the “active” sensing units 15_{i,j} (those for which the values are not previously discarded) at the comparison frequency Fm and this allows to decide on the “condition” of the sensing units 15i,j in a very short time.

The analysis method 200 further comprises an operating process 60 in which the calibrated data dc(i,j,t) are processed to obtain processed data dp(i,j,t), the operating process 60 comprising at least one the following steps: a fault-check process 55 that processes calibrated data dc(i,j,t) to obtain fault check data df(i,j,t), an adaptation process 56 that processes fault-check df(i,j,t) data to obtain adapted data da(i,j,t), a gait segmentation process 80 that processes adapted data da(i,j,t) to obtain the validated gait phase data Iv*(t).

In a version the adaptation process 56 processes the calibrated data dc(i,j,t).

In a further version the gait segmentation process 80 processes fault-check data df(i,j,t) to obtain the validated gait phase data Iv*(t).

In a further version the gait segmentation process 80 processes calibrated data dc(i,j,t) to obtain the validated gait phase data Iv*(t).

It should be understood that the fault-check process 55 and the adaptation process 56 may be performed also in a different order from what shown depending on the choice of the user.

The fault check process 55 evaluates if one or more sensing units 15 and/or one or more insole layers 12 of the insole are faulty and provides fault-check data df(i,j,t). The fault check process 55 is performed online and used in the operating process 60 to collect or send to the computing device 4 reliable data only, as explained below. The fault check process 55 also uses, if available, the comparisons with the second offload threshold TA2 and second onload threshold TC2 as an aid to identify the faulty sensing units and layers, as explained below.

The fault check process 55 is based on the principle that spatially-close sensing units 15i,j should yield similar data and, similarly, two data samplings close in time should yield similar values.

The closest two sensing units 15_{i,j} are, the highest their probability to return similar data. Furthermore, if sensing units are placed one on top of the other, mechanical deformations are expected to produce very similar data and only slight variations in pressure readings or response over time. In the fault check process 55 this principle is used for determining similarity in calibrated data dc(i,j,t) from different layers (i) in mutually corresponding positions, and from the same layer (i) in different positions (j) close one to another and over time (t) at interval Fwl, which is the fault window length as described later. The fault check process 55 can be either performed using calibrated data dc(i,j,t), or adapted data da(i,j,t) without changing the underlying principles.

In the fault check process 55 (see FIG. 13A), the processing device 23 calculates the coherence “C” of calibrated data dc(i,j,t), i.e. a factor indicating the similarity of calibrated data dc(i,j,t) in space (i,j) and over time (t) within a fault check time Tf. The fault check time Tf represents the overall interval of time over which the fault check process 55 is made, and it ranges from the current time t* to t*−Tfl in the past, for instance Tfl can be 10 s. The fault check time Tf is divided in a number Nwf of fault check windows Fwᵢ, i=1, 2, . . . , Nwf of length Fwl if expressed in seconds or Fwls if expressed in samples. If fault check time Tf is divided into Nwf=10 intervals (fault check windows), then Fwl=0.1*Tf and Fwls=Fwl*Fs where Fs is the sampling frequency of the calibrated data dc(i,j,t). An evaluation of possibly faulty sensing units 15_{i,j} and insole layers 12ᵢ is carried out for sampled data of each fault check window Fw, d(i,j,Fw) and it is performed over each different fault check window Fwᵢ of the fault check time Tf. The results obtained over the fault check windows Fwᵢ within the fault check time Tf are then evaluated to determine if a insole layer 12 is labelled as possibly faulty for more than fault layer threshold FLT of the total number Nwf of fault check windows within the fault check time Tf, as schematically indicated by FIG. 13B. The fault layer threshold FLT is a certain percentage, usually about 70%.

In the fault check process 55 the first onload threshold values TC1_{i,j} for each sensing unit 15_{i,j} are retrieved from the memory unit 24 and used to determine if a sensing unit 15_{i,j} is active or not, as explained in the following.

Owing to the fault check process 55, for each sensing unit 15_{i,j} it is determined if the sensing unit j of layer i, 15_{i,j} is active i.e. calibrated data dc(i,j,t) that are not above the first offload threshold value TA1 are discarded and not used in the fault check process 55.

This avoids that data samples of inactive sensing units influence the decision about the functioning of the sensing units 15_{i,j}.

The fault check process 55 comprises a fault calculation phase 55A in which the correlation “C” between the sampled data of each fault check window d(i,j,Fwi) of each pair of sensing units 15a,j and 15b,j (e.g., two sensing units, one on top of the other, along Z) is calculated so as to obtain a matrix Cab. The matrix Cab is symmetric and the coordinates (a,b) represent the correlation value (or coherence) between the data pertaining to the fault check window Fwᵢ and sensing unit j of layers a, b, that is d(a,j, Fwᵢ) and d(b,j, Fwᵢ) where a=1, . . . N, b=1, . . . N.

In this way, it is calculated the similarity of data sampled by sensing units 15_{i,j} positioned in corresponding positions in the different layers of the insole 1. If more than a certain number “g” elements of line “a” of coherence matrix Cab, except element Caa are below a fault threshold value TFa, the sensing unit 15_{a,j}, (sensing unit j of the layer a) is labeled as “possibly faulty”, as indicated in FIG. 13B. If more than “h” sensing units 15_{i,j} of one insole layer 12i are labeled as faulty, the insole layer 12i is labeled as “possibly faulty”. If a sensing unit 15_{i,j} is labeled as “possibly faulty” for a certain number of windows Fwᵢ out of the total it reaches the “faulty” status from the “possibly faulty” status.

The fault threshold value TFa, the value “g” of elements of a line at which the line is indicated as possibly faulty and the value of the number “h” of the sensing units of a layer at which the layer is indicated as possibly faulty, are stored in the memory unit 24 or provided by the producer of the system. The fault threshold value TFa is a threshold value based on the correlation of data. The value of the fault threshold value TFa depends on the mechanical properties of the system of the invention, i.e. for example of the sensing unit, on the type if footwear etc. The value of the numbers “g” and “h” indicated above, i.e. the number of possibly problematic values necessary to decide if the sensing unit 15_{i,j} or respectively the insole layer 12i is faulty may be decided by the user or by the manufacturer of the insole 1 and may vary according to the number of sensing units 15_{i,j} per insole layer 12i or insole layers 12 and it is connected with the robustness of the analysis system 100 and on the number of the layers and of the sensing units per layer.

The above indicated calculation of the coherence C is repeated for each fault check window Fwᵢ, as schematically indicated by the arrows in FIG. 13A, and if for more than fault layer threshold FLT of the total number Nwf of fault check windows, the insole layer 12i is labelled as possibly faulty, then the insole layer 12i is labelled as “faulty”, as indicated in the graph of FIG. 13C. In parallel, if the same single sensing unit 15_{i,j} is labelled as “possibly faulty” for more than a certain percentage of windows “fault sensing threshold” FST of the total number Nwf of fault check windows within the fault check time, then said sensing unit 15_{i,j} is labelled as “faulty”. The fault sensing threshold FST is usually about 80%.

In short, a insole layer 12i is labelled as “faulty” if more than “h” sensing units are faulty for more than a certain percentage of windows within the fault check time Tf.

The sensing units 15_{i,j} which are not considered faulty overcome the fault check process and the sampled data from these sensing units are indicated as fault-check data df(i,j,t).

The fault check process 55 comprises a further fault calculation phase 55B in which the calibrated data dc(i,j,t), transformed as previously discussed, are compared with the second onload threshold value TC2_{i,j} and discarded if higher (i.e., noisy). If after the further fault calculation phase 55B a sensing unit 15 is deemed faulty, it is included in the count of faulty sensing units 15_{i,j} and contributes with the other sensing units 15 to the labeling of insole layers 12 as “possibly faulty” and finally “faulty”, as explained below. This procedure is repeated for all the sensing units 15_{i,j} of the insole 1. In the further fault calculation phase 55B it is decided about the condition of the sensing units 15_{i,j} of the insole 1 using a sort of majority voting.

If, for example, on a column “j” all the sensing units 15_{i,j} but one give similar values over a certain period of time, the sensing unit 15 that gives a different a value will be labelled as possibly faulty, a corresponding signal is emitted and a corresponding decision, for example, not to consider that sensing unit 15 in the future is taken.

In the same way, if in a column “j”, or in a layer “i”, all the sensing units 15_{i,j} except one have a quantity of noise in frequency (highlighted by the second threshold values) below threshold, the sensing unit for which the noise is above threshold is probably the faulty one. Therefore, the provision of the threshold values allows to obtain fast and reliable indication about the condition of the sensing unit.

With reference to FIG. 13, graph 13B shows highlighted in gray some of the possibly faulty sensing units as an example. The one on the top left is labeled as “faulty” in this example because it is “possibly faulty” for all the fault check windows Fwᵢ represented in the picture, that is a percentage of fault check windows Fwi out of the total number of fault check windows Nwf, greater than the fault sensing threshold FST of 80%. FIG. 13C shows an histogram of the number of times each insole layer 12i is labeled as possibly faulty over the total number of fault check windows Nwf. As explained above, an insole layer 12i is labeled as possibly faulty in the fault check windows Fwᵢ because more than h sensing units in that layer (row in the matrixes C shown in panel 14B) are labeled as faulty. The fault layer threshold FLT above which the layer is considered faulty is represented by a horizontal line in FIG. 13C. The layers that are labeled as possibly faulty for a percentage over the total number of fault check windows Nwf greater than the fault sensing threshold FLT are labeled as faulty and in FIG. 13C are represented as greyed out.

In short, for deciding about the real condition of the sensing units 15_{i,j} or insole layer 12i it is not enough that the sensing unit 15_{i,j} and insole layer 12i result potentially faulty just once in a fault check window Fwi but they must result potentially faulty in a predetermined number of fault check windows Fwi. The data originating from sensing units 15_{i,j} or insole layers 12ᵢ that are labeled as faulty will not be considered further during the functioning of the system.

This implies that only useful data are used in the operating process 60, thus reducing the memory requirements and/or the energy spent and also reducing the response time of the analysis system 100 of the invention. These advantages are obtained while maintaining at the same time a high precision of the analysis system 100. The precision obtained is comparable or even greater than the precision obtainable with systems which require much more energy and/or much more memory storage.

In a version of the method of the invention it is provided for using also data from possibly faulty sensing units.

In the operating process 60 the fault check process 55 is performed on the calibrated data dc(i,j,t) from the evaluation phase 61, i.e. the data that have already been compared with the first offload threshold TA1_{i,j} and first onload thresholds TC1_{i,j,e} by means of the first threshold comparing process 50′A.

In the operating process 60, the fault check process 55 is also aided by the second threshold comparing process 50′B, that compares the sampled data d(i,j,t) with the second offload threshold TA2_{i,j} and second onload threshold TC2_{i,j}. In fact the second threshold comparing process 50′B provides already a list of faulty sensing units that can be combined to the list of faulty sensing units yielded by the fault check process 55 or used independently without the fault check process 55. In the version of the analysis method 200 of FIG. 9 the fault check process 55 is always active only in the onload condition. In the offload condition, the second threshold comparison process 50′B already yields a list of faulty sensing units. In the onload condition, the list of faulty sensing units given by the fault check process 55 is merged with the list of faulty sensing units yielded by the second threshold comparison process 50′B. The fault check process 55 further comprises a norm calculation phase 55C that provides for the calculation of the norm of coherence matrix Cab, i.e. the global coherence Q(Cab).

The global coherence Q(Cab) can be calculated as the sum of values, or another norm of the average matrix Cab over the fault check windows Fwᵢ within the fault check time Tf obtained in the first calculation phase 55A. A sudden or slow decrease in the absolute value of global coherence Q(Cab) is also used to detect a scenario in which a fault affects the whole system (e.g., a fault in the insole connector). Since, in this case, it is not possible to identify a particular faulty layer, a warning will advise the user to verify/replace all the sensing unit layers. The global coherence Q(Cab) also gives useful information about possible performance degradation. The global coherence Q(Cab) is calculated multiple times in different fault check times Tf and the values obtained are then compared. If the global coherence Q(Cab) decreases over time in different fault check times Tf for more than a certain fault check decay threshold Tfb (e.g., 30%) a performance decay is detected and the user is warned about a possible performance degradation which may lead to a fault.

The adaptation process 56 can be performed before a new use if any of the user, the footwear, or the task are changed or to adapt the stored threshold values to the real condition of use in order to adapt the analysis system 100 to the real condition of use. The adaptation process 56 allows to scale the calibrated data dc(i,j,t) or fault-check data df(i,j,t) by a factor based on the amplitude as will be explained in the following. This allows more precise information and more precise data from the system to be obtained. The adaptation process 56 is not mandatory but it is optional, except if voltage readings are required to be translated into actual weight estimates, e.g., when the user changes. Owing to the adaptation process 56 it is possible to adapt, i.e. reduce or increase, the emitted values taking into account the condition of use to obtain results responsive to the real situation. This avoids for example that an emitted value is overestimated or underestimated. If, for example, the onload threshold values are obtained based on a running, or walking activity in the production site and the analysis system 100 is, on the contrary, intended for being used for an obstacle race, it would be necessary to adapt the analysis system 100 to the actual activity to be performed.

In the adaptation process 56 it is created, for each sensing units 15_{i,j}, a linear or a nonlinear function that, if multiplied to the value i.e. the voltage emitted by the sensing units 15_{i,j}, will scale it to yield the actual weight. With the word weight it is hereto understood the ground reaction force at that particular site. The adaptation process 56 can be thus considered as a filter that transforms the calibrated data dc(i,j,t), or preferably fault check data df(i,j,t), into adapted data da(i,j,t) that can be used, instead of the calibrated data dc(i,j,t) in the operating process 60, as explained in the following.

In other cases it is provided for using a standard transfer function for adapting the analysis system to the new condition of use, nevertheless the provision of the adaptation process allows a more precise method to be obtained. The adaptation process 56 allows to scale the calibrated data dc(i,j,t) to the actual weight.

In the adaptation process 56 the data from the insole 1 are sampled for a predetermined period of time, indicated as adaptation measuring time, Ta. (FIG. 12)

For each insole layer 12i, and for each sensing unit 15_{i,j} in the insole layer 12i and for each weight available Wk, k=1, . . . , O including a null weight and the readings caused by wearing for instance a tight footwear, it is defined a multiplication factor “Q” so that, during use calibrated data dc(i,j,t) is divided by the appropriate multiplication factor Q to obtain the adapted data da(i,j,t). More specifically:

da(l,j,t)=dc(l,j,t)/Q_{i,j,k} formula 5

where k=1, . . . O is the weight used as explained below.

The weights available are the weights that ideally span the expected working range of the insole, for instance weights can be 5 Kg, 15 Kg, 25 Kg, . . . , 125 Kg. In the adaptation measuring time Ta, each weight available Wk is placed on the insole and the corresponding calibrated data dc(i,j,t) are recorded within an adaptation window Awₖ. The adaptation measuring time Ta is divided into some adaptation windows Awₖ each one corresponding to one of the available weights and the above indicated calculation is performed in each adaptation window. The adaptation measuring time Ta is divided into O adaptation windows Awₖ k=1, . . . , O, where “O” is the number of available weights, each adaptation window having a length given by Awls=Awl*Fs, where Fs is the sampling frequency, Awls the length of adaptation window in samples, Awl the length of the adaptation window in seconds.

Each adaptation window has length in seconds Awl of about 1 s and length in samples Awls and corresponds to the period of time in which a predetermined weight acts on the insole. Therefore, in each adaptation window there are obtained some calibrated data dc(i,j,t) the value of which reflects the action of the different weights on the insole 1.

The calibrated data dc(i,j,t) corresponding to the adaptation window Awₖ is indicated as d(i,j, Awₖ). For each adaptation window Awₖ the distribution of sampled data d(i,j, Awₖ) is taken into account and the following values are calculated: the lower (Dlow_{i,j,k}) and higher (Dhigh_{i,j,k}) range value, e.g. as the 10^{th} and 90^{th} percentile of the distribution of the data d(i,j, Awₖ), and the average Dmed_{i,j,k} value, for example the arithmetic mean of the sampled data d(i,j, Awₖ), however any other mean can be used. The multiplication factor Q_{i,j,k} relative to the adaptation window Awₖ is then calculated as follows:

Q_{i,j,k}=Dmed_{i,j,k}/Wₖ formula 6

where Q_{i,j,k} is basically the best estimate of the factor that divides the calibrated data dc(i,j,t) using Formula 5 above when calibrated data dc(i,j,t) is within the range defined by the interval [Dlowₖ Dhighₖ] and it yields adapted data according to the following formula 5.

The values of the adapted data da(i,j,t) are closer to the actual weight Wₖ. At the end of the adaptation process 56, a matrix of multiplication factors Q_{i,j,k} and corresponding intervals [Dlow_{i,j,k}, Dhigh_{i,j,k}] are created for each sensing unit 15_{i,j} and weight Wₖ. These constitute the transfer function that is used for transforming the values emitted by the sensing units 15_{i,j} (voltage) to reach the actual weight applied to each the sensing unit 15_{i,j}.

During normal use, the multiplication factor Q_{i,j,k} divides the calibrated data dc(i,j,t) only when the calibrated data dc(i,j,t) falls within the range defined by the interval [Dlow_{i,j,k} Dhigh_{i,j,k}]. These intervals are calculated for each weight Wₖ.

It is possible that there are ranges of value of calibrated data dc(i,j,t) that do not fall within any interval [Dlow_{i,j,k} Dhigh_{i,j,k}]. In this case the data are divided by a multiplication factor Q that is the combination of the multiplication factors Q_{i,j,k} belonging to the closest intervals.

For example, if the value of calibrated data dc(i,j,t) is higher than Dhigh_{i,j,k} and lower than Dlow_{i,j,k+1} then the following formula 7 is used for calculating adapted data:

da(i,j,t)=dc(i,j,t)/Q_{MEDk} formula 7

where the multiplication factor Q_{MEDk}=(Q_{i,j,k}+Q_{i,j,k+1})/2, as indicated in case 1 of FIG. 12B).

The same calculation is done if the value of the calibrated data dc(i,j,t) falls within two intervals, for example if it is lower than Dhigh_{i,j,k} and higher than Dlow_{i,j,k+1}, as shown in case 2 of FIG. 12B). Also in this case Q_{MEDk}=(Q_{i,j,k}+Q_{i,j,k+1})/2. A standard matrix of multiplication factors Q_{i,j,k} and intervals [Dlow_{i,j,k}, Dhigh_{i,j,k}] may also be provided to the user, based on factory assessment for the different condition of use, or weights, of the user. The adapted data da(i,j,t) obtained with Formula 5 take into account the different condition of use of the different sensing units 15_{i,j} of the insole 1, of the strains differences of the different sensing units 15_{i,j} causing different voltage output i.e. emitted data D(i,j,t).

The adaptation process 56 also allows to homogenize the sampled data values of the sensing units 15_{i,j} in corresponding positions and different layers 12_{i.}

The adaptation process 56 is made by the acquisition device 2 or by the computing device 4. The adaptation process 56 can be performed every time it is deemed necessary and allows array of adapted data da(i,j,t) to be obtained. The adapted data da(i,j,t) of the adaptation process 56 are stored in the memory unit 24 of the acquisition device 2 and can be used in the operating process 60, as explained in the following. The adaptation process 56 allows the precision of the analysis performed by the analysis method 200 to be kept high.

The operating process 60 of the analysis method 200 is performed after the calibration process 50 is finished, when the latter is provided in the method 200. In case the calibration process is not provided in the method of the invention, the operating process 60 is performed using threshold values stored in the memory unit 24, for example defined at the production site of the insole 1.

The processed data dp(i,j,t), i.e. the value which have passed the evaluation phase 61 and/or the fault check process 55, and/or adaptation process 56 go then into a memorization process 62 along with gait data Iv*(i,j,t) and are memorized in the memory unit 24 and/or sent to the computing device 4.

Note that ideally the sampled data d(i,j,t) goes through all the aforementioned processes: calibration process 50, fault check process 55, adaptation process 56, gait segmentation process 80 but in some versions of the method some of the processes like fault check process 55, adaptation process 56, gait segmentation process 80) can be omitted.

During use the user wears the insole 1 connected to the acquisition device 2 and fastens the acquisition device 2 to the footwear or to the leg and begins the tasks he desires to accomplish.

The sensing units 15_{i,j} of the insole 1 continuously sense the pressure exerted by the user and send emitted data D(i,j,t) to the acquisition device 2. The processing device 23 of the acquisition device 2 samples from the emitted data D(i,j,t) the sampled data d(i,j,t) at the sampling frequency Fs.

The analysis method 200 of the invention further comprises a gait segmentation process 80 allowing the gait of the user to be segmented and to be analyzed in a more precise and faster way compared with the known analysis systems. The gait segmentation process 80 is performed on calibrated data dc(i,j,t) or preferably fault-check data df(i,j,t) or more preferably adapted data da(i,j,t). In fact, better performance of the gait segmentation process 80 is achieved by using sensing units 15_{i,j} and insole layers 12i of the insole 1 that are not tagged as faulty.

The gait segmentation process 80 comprises a gait calculation phase 81 in which calibrated data dc(i,j,t) are evaluated as explained below and a gait evaluation phase 82 in which the data obtained in the gait calculation phase 81 are sent from the acquisition device 2 and displayed indicating the gait phase and/or stored in the memory unit 24.

For activating the gait segmentation process 80, the user selects the modality of use, for example run, sprint, walk, etc. and for each modality of use the corresponding set of matrices MwL relating to that task, each weight matrix Mwl of the set of matrices MwL containing the distributions of weights for each gait phase I of the task. The matrices of the set of matrices MwL are stored in the memory unit 24 of the acquisition device 2. The sensing units 15_{i,j} with j=1, . . . M of the same insole layer (12i) have for each gait phase I a different level of activation, i.e. a different amplitude of the calibrated data dc(i,j,t) and yield the maximum value only when multiplied with the appropriate weight matrix Mwl corresponding to that gait phase I.

If a sensing unit 15_{i,j} of insole layer 12i is faulty then the corresponding calibrated data dc(i,j,t) is set to zero and this sensing unit 15_{i,j} is not considered. Analogously, if a sensing unit 15_{i,j} of layer “i” is not active, i.e. the current calibrated value dc(i,j,t*) is below the first offload threshold value TA1, then its value is set to zero. Clearly, if too many sensing units 15_{i,j} in a certain area of the insole 1 are faulty, then it is not possible to correctly evaluate the gait phase I and a corresponding “not availability” signal is emitted and/or displayed.

The number of not existing sensing units 15_{i,j} for deciding on the “not availability” of data may be determined by the user and/or during the manufacturing of the acquisition device 2. According to the gait segmentation process 80, for each possible task, like for example walking, running, obstacle race, etc., it is created a predefined set of weight matrices MwL. Each weight matrix Mwl with I=1:L, of the set of weight matrices MwL indicates the supposed level of activation of each sensing unit 15_{i,j} in a gait phase I of the task. Each weight matrix Mwl has a number of elements equal to the number of sensing units 15_{i,j} of the insole 1.

The number of the phases L, that corresponds to the number of weight matrices Mwl within a set of weight matrices MwL and that corresponds to the number of phases in which the gait of the task is divided is chosen by the user and may depend, for example, on the type of task performed. Typically L can range between 2 and 20 and is preferably 5. The different weight matrices Mwl act as spatial filters on the calibrated data dc(i,j,t) obtained by the sensing units 15_{i,j} and allow to reliably detect the gait phase I. Each weight matrix Mwl comprises values between minimum (0) and maximum (1), the number of ranges in which the interval is divided depends on the task performed and on the precision required. For example, in the case of the gait phase I corresponding to heel strike, the weight matrix Mwl is so configured that there are high values (i.e., closer to 1) for the sensing units 15_{i,j} positioned in the heel part 1B of the insole 1, and low values (i.e. closer to 0) for the sensing units 15_{i,j} positioned in the toe part 1A of the insole 1.

During the gait segmentation process 80, at the current time instant t*, the calibrated values dc(i,j,t*) from each sensing unit 15_{i,j} are respectively multiplied for all the weight matrices Mwl of the preset set of matrices MwL for the task which is performed, i.e. for the matrices of all the possible phases in which the task being performed has been divided. With these multiplications it will be obtained a set of weight multiplication values ML. The actual gait phase “I” is the one for which the multiplication value MI of the set of weight multiplication values ML is maximum. The actual gait phase I* is the phase for which the values distribution across sensing units 15_{i,j} is matching the distribution of weights given by a predefined weight matrix Mwl. The multiplication value MI is maximum when the current calibrated value dc(i,j,t*) are multiplied for the right weight matrix Mwl, i.e., the one corresponding to the actual gait phase I*, i.e. the weight matrix Mwl having the highest value in correspondence of the sensing units 15_{i,j} having the highest level of activation. The multiplication values MI of the set of weight multiplication values ML are compared one with another and the greatest one is selected, the one which has the higher number of maximum values.

Said maximum multiplication value is indicated as MI_{MAX} and corresponds to the actual gait phase I*. At a single calculation it is selected the phase I* corresponding to the matrix Mwl giving the highest value in the multiplication. For example, in the case of the phase I* corresponding to the heel strike, the maximum multiplication value MI for the corresponding weight matrix Mwl is obtained only when the sensing units positioned in the heel part 1A of the insole 1 are pressed. When this happens the gait phase I* is labelled as “heel strike”.

The maximum multiplication value MI_{MAX} is made available with a very short delay and provides information about the gait phase I*. Since this calculation is performed ideally at the same sampling frequency Fs, the actual gait phase I* is actually a signal I*(t). By analysing the actual gait phase I*(t) over time, information about the duration of the single gait phases can be inferred.

The steps hitherto described form the gait calculation phase 81 in which the output is the actual gait phase over time I*(t). The number L of weight matrices Mwl of the set of weight multiplication values MwL depends on the different task which could be examined with the analysis system 100 and/or method 200 of the invention. The different sets of weight matrices MwL are preloaded in the acquisition device 2, but, depending on the specific requirements of the user, the number of sets can be increased or updated.

In the gait evaluation phase 82, further to the gait calculation phase 81, the actual gait phase I*(t) is evaluated over time to become validated gait phase data Iv*(t). In the gait evaluation phase 82, the actual gait phase I*(t) is evaluated for a gait evaluation interval Tg. The length of the gait evaluation interval Tgl is chosen in seconds on the basis of the task under examination. If, for example, the activity is “running”, the length of the gait interval Tgl can be 0.1 s, if on the contrary the task is slower, such as “walking”, the length of the gait interval Tgl could be 0.2 s. Similarly to what described before, the gait interval Tg spans the interval of time [t*−Tgl, t*] that is the interval of length of the gait interval Tgl ending at the current time t*.

For deciding the validated gait phase data Iv*(t*) at the time t* a simple majority voting is applied on the actual gait phase I*([t*−Tgl, t*]). For example, if during the interval of time [t*−Tgl, t*], 15 times out of 20 the actual gait phase I* is derived as “heel strike” by the gait calculation phase 81 and the remaining 5 times out of 20 the actual gait phase I* is derived as “toe on”, then the validated gait phase data Iv*(t*) at the time t* is “heel strike”. Such majority voting can be weighted by the time distance with respect to the current time t*. For example, the actual gait phase Tgl seconds in the past I*(t*−Tgl) may be weighted 0.1 and be weighted up to 1 the closer it gets to the current actual gait phase I*(t*). In other words, the recognition of the actual gait phase I*(t) is “filtered” over time to increase the stability of the gait segmentation process 80. The extension of the gait interval Tg can be chosen by the user and it depends on the precision required by the method of the invention 200. It is also possible to adjust the weighing of the values obtained in the interval Tg.

The selection of the dimension of the gait interval Tg impacts the responsiveness in the conclusion about the validated gait phase data Iv*(t). In fact, the higher the interval Tg the more time it takes to recognize a change in phase (e.g., from “heel strike” to “toe off”) through majority voting. These parameters can be set as default and then chosen by the user.

If the majority voting is inconclusive, e.g., two phases have equal score or occurred the same number of times during the gait evaluation interval Tg, it is possible to set the gait phase as “possibly not available” (PNA) or randomly select one of the two possible phases.

It is further possible to analyze the succession of validated gait phase data Iv*(t), by setting up predefined sequences that are selected according to the condition of use chosen by the user. For example, it is more probable, during normal gait, that a “toe on” phase follows the “heel strike phase” than vice versa. If the sequence of phases is not as expected a warning may be given to the user. The validated gait phase data Iv*(t) is sent to the operating process 60 along with the fault-check data df(i,j,t) if available, and the calibrated data dc(i,j,t) and the adapted data da(i,j,t) if available.

The validated gait phase data Iv*(t) obtained at the end of the gait evaluation phase 82 are sent to the operating process 60 and stored in the memory unit 24 and/or sent to the computing device 4 and possibly displayed.

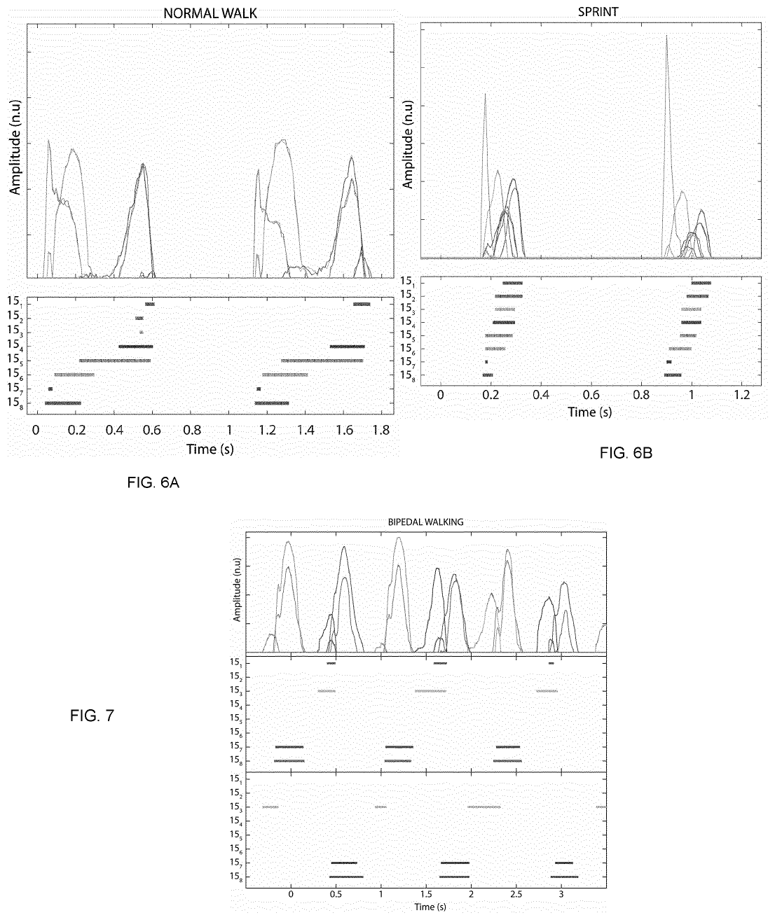

The result of the gait segmentation process 80 is shown for example in FIGS. 6A, 6B and 7. FIGS. 6A, 6B and 7 show how the scheme of the left/right foot integration layer that enables to observe gait differences between steps, between and within each side. FIG. 6A represents data obtained during normal walk, whilst FIG. 6B represents data obtained during sprint. Such data are obtained from one foot in two different gaits. Considering FIG. 6A it is indicated the activation of the 8 sensing units 15₁₋₈ over an interval of time. It is possible to immediately understand how the weight of the user is distributed over the different sensing units over time, i.e. if there is an asymmetry in the distribution of the weights. Comparing the two bar graphs of FIG. 6A it is also possible to understand if there are any differences between the two gaits. The Line Graph in the upper part of FIG. 6A reports the same information using continuous lines.

It is immediately apparent that the processing of the data with the gait segmentation process 80 of the invention allows to increase the responsiveness of the analysis method 200 of the invention and to furnish information with very short delay and also furnish information in a very user-friendly way. Same considerations will also apply to FIG. 6B.

Regarding FIG. 7, in the middle bar graph it is reported the degree of activation of the sensing units 15_{i,j} of two feet of the same user over time, in the lower graph bar the differences between the sampled data of the two feet are reported. A maximum 1 ms misalignment is ensured, if the two insoles are wired. It is possible to understand the activation level of the sensing units 15_{i,j} over time and if any asymmetry between the two feet is present.

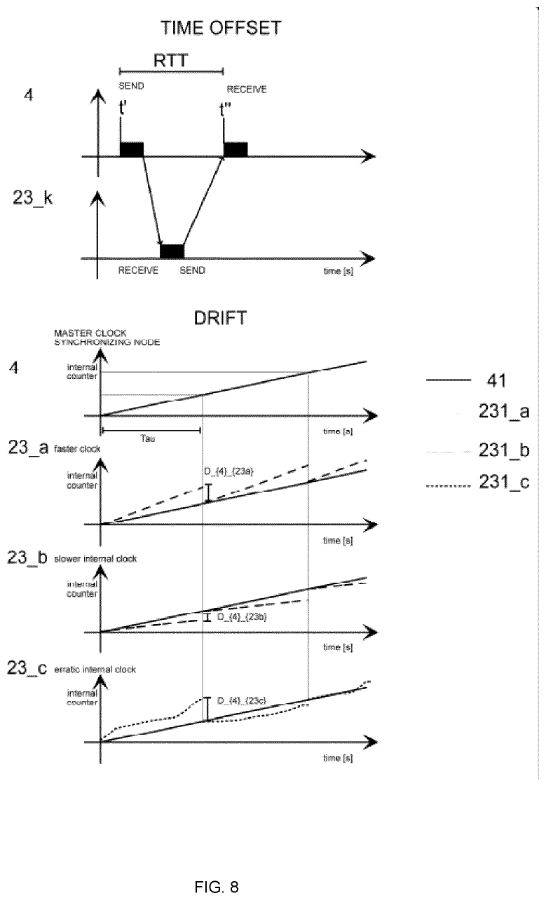

In FIG. 8 it is reported the Synchronization architecture for synchronizing two or more insoles. This is necessary for instance when performing gait segmentation on two feet while walking. Synchronization is necessary to correct for drifts in sampling frequency Fs between the different devices e.g., due to heating or other environmental issues. Synchronization allows to account for unpredictable environmental effects or possible waiting periods due to the various calculations performed. Small differences due to the internal characteristics and to local environmental characteristics such as e.g. temperature and humidity cause the internal clock 41 and the further internal clock 231 to vary at different, not necessarily constant, speed.

For this reason, over a prolonged acquisition time, the clock speed drift between the internal clock 41 and the further internal clock 231 may become not negligible. This issue is replicated with different local drifts between internal clock 41 of the computing device 4, and each of the further internal clocks 231 of the processing devices 23_a, 23_b, . . . , 23_n connected to the computing device 4. To mitigate this effect it is provided for synchronize the further internal clocks of the processing device as follows:

TIME OFFSET: The time t′ as measured from the computing device 4, is emitted as a message from the computing device 4 to the processing device 23, processed by the processing device 23 and then emitted from the processing device 23 and received by computing device 4 at the time t″. The interval of time between t′ and t″ is defined as the return travel time (RTT).

DRIFT:

In a defined time interval TAU [seconds], the uncorrected drift D_{4}_{23k} [seconds] between the computing device 4 and each of the processing devices 23_k is assessed.

The maximum of the absolute drifts D where D=max(abs(D_{4}_{23k})) [seconds] is used to define the minimum frequency at which a resynchronization signal from computing device 4 to the plurality of processing devices 23_1 . . . 23_n should be emitted/broadcasted.

Once defined MAD [seconds] as the maximum allowed drift, the resynchronization signal needs to be emitted at least every MAD*TAU/D [seconds]. In the hypothesis that the maximum clock drift is D=0.1 s in a time interval TAU=10 s, and MAD=0.001 s, the broadcasted synchronization signal needs to be sent at least every 0.001*10/0.1=0.1 seconds

The steps of the analysis method 200 of the invention can be summarized as follows.

The analysis system 100 is calibrated by means of the calibration process 50 and the threshold values of the sensing units 15 for the different operating conditions TA1, TA2, TC1e, TC2 are calculated and stored in the memory unit 24 of the acquisition device 2 and/or sent to the computing device. Alternatively, the different threshold values are already provided to the user by the manufacturer.

During use, the calibration process is constantly performed using the available thresholds values.

During use, in parallel to the calibration process 50, the fault check process 55 is also performed so as to evaluate and identify any faulty sensing unit and/or layer. The fault check process may use the information coming from the calibration process to increase the precision of the fault identification.

If some sensing units are deemed faulty, the corresponding sensing units are labelled accordingly and either discarded or considered for future evaluation and a corresponding warning signal is sent to the computing device 4 and possibly displayed in the display unit 6. The data coming from faulty sensing units is preferably not used in the adaptation and gait segmentation processes. If all the sensing units/layers are deemed faulty a corresponding warning signal is sent to the computing device and displayed in the display unit 6. If the number of faulty sensing units is too high the warning signal is sent as indicated above and the user knows that the analysis system cannot be properly used. If the fault check process 55 is unsuccessful warnings or blocking errors are raised or also alarms are produced by the acquisition device 2.

Furthermore, if necessary, the adaptation process 56 is performed for adapting the analysis system 100 to the actual condition of use. If the adaptation process 56 is not performed the analysis system 100 can be operated using pre-stored weights, either by default from producer or from last use, or last existing/matching calibration settings are loaded.

To put the analysis system 100 into operation, the user selects the using condition in the acquisition device 2 and begins the operating process 60.

In the operating process 60 the processing device 23 of the acquisition device 2 samples the sampled data d(i,j,t) from the emitted data D(i,j,t) from the insole 1 and may transmit via suitable further connection means 5, i.e. wifi or wired network, its ID to the computing device 4 and once secure communication is established the acquisition device 2 transmits sampled data d(i,j,t) and/or processed data dp(i,j,t) at the sending frequency Fp to the computing device 4. In detail, in the evaluation phase 61, the processing device 23 of the acquisition device 2 receives emitted data D(i,j,t) from the sensing units 15_{i,j} of the insole 1 and samples the sampled data d(i,j,t) at the sampling frequency Fs and processes sampled data d(i,j,t) to obtain calibrated data dc(i,j,t) that are sent to the operating process 60 and processed to obtain processed data dp(i,j,t) and validated gait phase data Iv*(t) that are stored in the memory unit 24 and/or sent to the computing device 4 or discarded.

The processed data dp(i,j,t) are transmitted or stored and labelled so as to contain information about the sensing unit 15_{i,j} that produced that data and/or about the activation thereof and is shown on the display unit 6 of the computing device 4.

The processed data dp(i,j,t) transmitted and/or stored are labelled so that the user knows which sensing units 15_{i,j} are active or not and can retrieve information on fault sensing units and also on the gait phase. Owing to the operating process 60 made by the processing device 23 of the acquisition device 2 only processed data dp(i,j,t) and gait-validated data Iv*(t) are sent to the computing device 4, and then displayed thereon. The preprocessing action of the processing device 23 allows to filter the emitted data and to send to the computing device only useful data, i.e. data coming from active sensing units. All data produced from non-active or faulty sensing units are not sent to the computing device. This implies that the communication between the acquisition device and the computing device is faster and more reliable. This also implies that all the data received by the computing device 4 are useful data. Therefore, it is not necessary to check in the acquisition device 2 and to process them before displaying them on the display unit 6, i.e. before making the transmitted data available for a user. This implies that data are ready substantially without delay for the user.

Therefore transmitted data are immediately available for further use. Data sent to the computing device 4 are sorted and processed by a software contained in the computing device to yield complex visualizations as well as real-time and offline analyses. Additionally, the fault check process 55 is continuously run by the acquisition device 2 with different thresholds values. This implies that if a sensing unit is faulty or a layer of sensing units is faulty the data therefrom are not used in the comparison phase, and will not affect the value of transmitted data. In this way, the acquisition device 2 guarantees robustness to faults of one or more insole layers, as long as at least 2 insole layers are still functioning, and detection of slow decrease in performance, fault prediction.

The sampled data d(i,j,t) are also processed to obtain information about the level of activation of the sensing unit and the gait phase, as indicated above. The analysis method of the invention also provides comprehensive visualization capabilities. The method may be run on various operating systems (OSs) for standalone applications or integrated in existing ones. It can run on a server-client basis and accessed via webpage-based client. The method may be used as a standalone application or as a platform for real-time data visualization, high-level data processing, device control (start, stop, modality selector etc.).

Owing to the method of the invention many advantages can be obtained like for example visualization of raw data (time series over time) with a selector for channels, visualization of processed data in the time and/or frequency domains visualization of gait phases, complex visualizations such as waterfall plot representation of the load.

The method also allows high-level data processing including detection of performance decay, analysis of the center of pressure and load distribution, assessment of gait, based on standardized features.

The analysis system and the analysis method of the invention allow many advantages to be obtained over existing systems. The sensing device 1 is redundant to ensure that the recorded pressures are similar, thus providing information about sensing units' wear-out or damage due to use.

The analysis system 100 is also able to perform walk segmentation and real-time classification of the contact phase during walking and running tasks. Owing to the system of the invention it is possible to assess the gait capabilities of humans in healthy conditions or with motor deficits, and to quantify the spatial distribution of loads, and the temporal patterns of loading and unloading of the foot, while minimizing the disturbances under the insole, and allowing to record the motor response (kinematic, kinetic and inertial) of the subject in relation to external triggered events or as a stream of data.

The analysis system of the invention is suitable for being used in many fields, like for example the ones listed below:

Robot-assisted-therapy: it enables highly intensive physiological gait rehabilitation for severely impaired neurological patients (e.g., after stroke or affected by Parkinson), thus allowing to define a personalized metric for optimal exploitation of neuroplasticity and recovery potential. For therapy to be effective however, reliable gait segmentation is necessary to control the robotic device and adapt gait patterns accordingly.

Mobile Brain/Body Imaging: monitoring brain plasticity via imaging techniques such as electroencephalogram enables to assess the recovery progress of the patient and give feedback to clinicians that allows them to optimize the therapy. Given the high speed of brain-muscle control and coordination, precise gait segmentation is required.

FES for restoration and rehabilitation: fast and reliable gait segmentation enables to identify the exact phase when to deliver functional electrical stimulation (FES) to maximize the rehabilitation therapy effectiveness or for restoration (e.g., Tibialis Anterior stimulation).

Feedback for prostheses control: using Transcutaneous or Intraneural stimulation it is possible to provide amputees with tactile feedback on foot ground contact. Ground reaction force needs to be estimated quickly and reliably.

Sport training: thanks to ground reaction force estimation during sport activities (e.g., running, long jump, high jump, obstacles etc.) it is possible to optimize athletic training, performance and give constant feedback to trainers. Examples are as follows:

Obstacles: evaluation of balance before the jump; evaluation of flight time over the obstacle;

Long jump: evaluation of spring and reactivity of the preparation run;

High jump: evaluation of push force of the last step;

Marathon, running: evaluation of stepping frequency and optimization of running patterns;

Evaluation in sports to assess the movement and the distribution of load of the performers;

Evaluation of postural balance: Benchmark of postural balance during assessment and rehabilitation exercises in gyms.

Footwear evaluation: footwear design evaluation based on feedback and wearability. It is possible to benchmark comfort by analyzing gait patterns of users wearing the footwear.

Foot ulcers for diabetics: feedback on prolonged localized loads has to be given to users having low to inexistent afferent information

The system of the invention has a very high reactiveness. The most stringent requirement for response time is given by “Feedback for prostheses control” and “FES for restoration and rehabilitation” applications. Considering a worst-case scenario of a trans-tibial amputation of 20 cm and a range of conduction velocity of peripheral nerves (medial/lateral plantar, tibial, saphenous), the device needs a response time of 3 ms. If this requirement is met, a stimulator can promptly deliver neuromorphic intraneural stimulation to provide feedback to the amputee on ground contact of the prosthesis without perceptible delay. All other applications have more lax requirements. The system of the invention has a high sampling rate. The most stringent requirement for wireless delivery sampling rate is given by the “Mobile Brain/Body Imaging” application. The required time resolution for effective event-related potential, time-frequency and connectivity analyses ranges can be considered 3 ms. Additionally, the analysis system 100 of the invention is robust and light and it is suitable for being used in sports applications in which the most stringent requirement in terms of robustness and lightness are requested.

Additionally, the analysis system 100 of the invention is suitable to be used as a standalone product or it can be integrated with other devices. The analysis system 100 of the invention is very reactive, as it has a max 2 ms delay.

The analysis system 100 of the invention performs an assessment of the performance decay, assessing the functioning of the sensing units 15_{i,j} of the insole 1 and advises on the necessity to substitute the sensing unit(s) and/or the insole 1.

In view of the high number of sensing units 15_{i,j} used, the sensing device 1 ensures redundancy. This allows a real-time coherence analysis that allows to detect failures and advise the user on the necessity to substitute the sensing device 1. The sensing device 1 works also in case of failure of one or a certain number of sensing units 15_{i,j}.

The sensing device 1 is light and thin (1-2 mm maximum), this implies that there are no constraints on the footwear worn. Additionally, the acquisition device 2 is less than 40 g weight so the user does not feel any additional weight.

The analysis system 100 of the invention further allows to synchronize signals generated by a plurality of acquisition devices 2. Synchronization of a plurality of acquisition devices 2 is particularly relevant in some applications.

For example, synchronization of data generated by the two feet while walking allows a more precise gait analysis, e.g., analysis of swing and stance phases for each leg. This allows to study e.g. gait symmetricity or the length of the double support phases. Also, for prosthesis control, synchronization allows to use information retrieved from one foot to aid with the other.

The synchronization is also important for monitoring different users at once for example for medical, sports, training, fitness, and entertainment applications. The synchronization would prove extremely useful for monitoring the performance of users moving independently to benchmark their performance for example while playing basketball, soccer, running or patients walking inside an hospital or in a garden. It would be also possible to benchmark coordinated group activities where synchronized execution of a task is important. Owing to the very short delay in the communication between the acquisition device 2 and the computing device 4, it is possible to effectively synchronize a plurality of acquisition devices 2.

The acquisition devices 2 to be synchronized must be positioned in a spatial range in which they can communicate data to the same computing device 4. The synchronization between two wearable analyzing devices 100A may be obtained in a wired mode or also in a wireless mode.

In wired mode, the synchronization may be obtained by connecting the two acquisition devices 2 by means of a synchronization cable not visible in the Figures.

In wired mode, the two acquisition devices 2 are connected by the synchronization cable and switched on, a sensing unit of one of the acquisition devices 2 generates a periodic clock signal used to trigger the start of the execution of one frame of code on the connected embedded systems. The trigger is implemented with, but not limited to, transistor logic (TTL, LVTTL, etc. . . . ) and a positive—or negative—edge is used as a synchronization signal to start the execution of one frame of code on each of the acquisition devices. The trigger can also be implemented with serial or parallel communication to include parameters apt to define or change the executed code in the triggered frame or in all and any future triggered frames. In wireless mode, the acquisition device 2 periodically transmits an activation signal, i.e. a datagram to the computing device 4 that triggers the synchronization. The process is obtained by explicitly targeting one or more acquisition devices sequentially with a common or personalized datagram, or by broadcasting a common datagram. On the receiving end of the network, successful recognition of a trigger can be determined by an incoming packet on a predefined port, or by the content of the datagram in full or of a subset. For both wired and wireless communication, on the acquisition device a listener detects incoming information and:
triggers transmission of the data sampled during the previous frame;
if the triggering event does not contain dynamic reconfiguration information or dynamic reconfiguration is not enabled—via software or hardware configuration—on the sensing unit node hardware, pre-existing frame configuration is used;
if the triggering event supports a datagram, or equivalent information, and the execution mode supports dynamic reconfiguration, the incoming frame configuration parameters of the datagram are read, and validated against sensing unit node type and matching IDs. If the masked information applies to the sensing unit node, the frame configuration on the device is updated.
if a valid frame-parameters reconfiguration is detected, the corresponding hardware and timing properties are updated. Configuration information can include: programmable hardware gain for each sampled channel, subset of channels to be sampled, switching and sampling delay between channels in multiplexed systems, oversampling factor on reading sensing units data, sampling delays for chunk reading, parameters for onboard averaging and filtering and subsampling of each channel data. Configuration status is returned.

If a valid configuration status is confirmed, sampling of the sensing units is performed in accordance with the configuration, and on-board processing as described in section 5.2 is performed. At frame completion, frame data are transferred to the network communication buffer (netcomm) and to the logger buffer, then frame sampling is acknowledged as completed.

1. Analysis method (200) for analyzing pressure exerted by a portion of the body and/or of a human prosthesis comprising:
sampling data generated by the portion of the body and/or the human prosthesis during a threshold calibration period (TCP) at a sampling frequency (Fs) in at least one condition of use so as to obtain an array of sampled data (d(i,j,t)) of at least one sensing unit (15_{i,j}),
providing at least one threshold value (TA1, TC1) of the sampled data (d(i,j,t)) for the at least one condition of use and storing the least one threshold value (TA1, TC1) in a memory unit (24),
comparing at the sampling frequency (Fs) the sampled data (d(i,j,t)) with the at least one threshold value (TA1, TC1) to obtain calibrated data (dc(i,j,t)), and
storing the calibrated data dc(i,j,t) in the memory unit (24) and/or sending the calibrated data dc(i,j,t) to a computing device (4) if the calibrated data dc(i,j,t) is greater than the at least one threshold value (TA1, TC1).

2. The method according to claim 1 further comprising an operating process (60) in which the calibrated data (dc(i,j,t)) are processed to obtain processed data (dp(i,j,t)), the operating process (60) comprises at least one the following steps: a fault-check process (55) that processes calibrated data (dc(i,j,t)) to obtain fault check data (df(i,j,t)); an adaptation process (56) that processes calibrated data (dc(i,j,t)) or fault-check (df(i,j,t)) data to obtain adapted data (da(i,j,t)); or a gait segmentation process (80) that processes calibrated data dc(i,j,t), or fault-check data df(i,j,t) or adapted data da(i,j,t) to obtain validated gait phase data (Iv*(t)).

3. The method according to claim 2 further comprising an evaluation phase (61) in which sampled data (d(i,j,t)) are processed to obtain calibrated data (dc(l,j,t)) that are then sent to the operating process (60).

4. The method according to claim 1, further comprising providing at least one offload threshold value (TA1) for an offload condition in which the portion of the body and/or the human prosthesis does not exert any pressure, and/or at least one onload threshold values (TC1) for an onload condition when the portion of the body and/or the human prosthesis exerts a pressure, when user is in a moving condition performing a task.

5. The method according to claim 4, further comprising providing a set of first onload threshold values (TC1E) for the moving condition, each of the first onload threshold value (TC1e) of the set of first onload thresholds values (TC1E) corresponding to a different level of activation of the at least one sensing unit (15_{i,j}).

6. (canceled)

7. (canceled)

8. (canceled)

9. (canceled)

10. The method according to and claim 1, further comprising a calibration process (50, 51, 53) in which at least one threshold value (TA1, TA2, TC1, TC2) of the sampled data (d(i,j,t)) for at least one condition of use is calculated (51, 53) and stored in a memory unit (24).

11. The method according to claim 10, wherein said calibration process (50, 51, 53) comprises a first threshold calculation step (511, 531) in which the first threshold value (TA1, TC1) of the sampled data for the at least one condition of use is calculated, the first threshold calculation step (511, 531) is performed on a threshold recording period (Rp) and comprises dividing the threshold recording period (Rp) in a plurality of recording windows (Rwᵢ) and calculating the maximum of the sampled data d(i,j,Rwi) at each recording window (Rwᵢ) thus obtaining a first threshold value of the recording period (Ta1(Rwᵢ)) for the at least one condition of use and further calculating a desired percentile threshold of the first threshold values (Ta1(Rwᵢ), i=1, . . . , nW), 95-99%, of the first threshold values (Ta1(Rwᵢ)) and adding a safety margin thus obtaining the first threshold value (TA1(Rpᵢ)) for the at least one sensing unit (15_{i,j}) and each threshold recording period (Rpᵢ) and the at least one condition of use.

12. The method according to claim 11, wherein said calibration process (50, 51, 53) comprises a second threshold calculation step (512, 532) in which a second threshold value (TA2, TC2) for the at least one condition of use is calculated, the second threshold calculation step (512, 532) is performed over a threshold recording period (Rp) and comprises dividing the threshold recording period (Rp) in a plurality of recording window (Rwi) and calculating a second threshold value in each recording window Rwᵢ (Ta2(Rwᵢ)) according to formula 3:
Ta2(Rwi)=k0|d(tf)−d(tf−1)|\hat{ }n+k1|d(tf−1)−d(tf−2)|\hat{ }n+ . . . kp|d(2)−d(1)|\hat{ }n (formula 31
thus obtaining a second threshold value of the recording period (Ta2(Rwᵢ)) for the at least one condition of use and further calculating a certain percentile threshold of the second threshold values (Ta2(Rwᵢ)), preferably 95-99% of the second threshold values (Ta2(Rwᵢ)) and adding a safety margin thus obtaining the second threshold value (TA2(Rpᵢ)) for the at least one sensing unit (15_{i,j}) and each threshold recording period (Rpᵢ) and the at least one condition of use.

13. The method according to claim 12, wherein the calibration process (50) comprises a validation process (54) for checking if the first and/or second threshold values (TA1(Rpᵢ), TA2(Rpᵢ)) obtained by the first and/or second threshold calculation step (512, 532) respectively are stable over time and/or spatially and for calculating and storing a mean value of the first and/or second threshold values (TA1, TA2, TC1, TC2).

14. The method according to the claim 13, wherein the validation process (54) comprises a first threshold validation step (541) for checking if the multiple first and/or second threshold values (TA1(Rpi), TA2(Rpi)) are stable over time, the first threshold validation step (541) provides for performing the first (512, 532) and/or second threshold calculation step (512, 532) multiple times over consecutive threshold recording periods (Rp) so as to obtain multiple first and/or second threshold values (TA1(Rpi), TA2(Rpi)) and for comparing multiple first and/or second threshold values (TA1(Rpi), TA2(Rpi)), if the multiple first and/or second threshold values (TA1(Rpi), TA2(Rpi)) are within a defined variability range (S) it is provided for calculating a mean value of the first and/or second threshold values of the recording period (TA1(Rpi), TA2(Rpi)) and storing the mean value as first and/or second threshold value of the at least one condition of use (TA1, TA2, TC1, TC2) in the memory unit (24).

15. The method according to claim 14, wherein the validation process (54) comprises a second threshold validation step (542) for checking if the first and/or second threshold values (TA1, TA2, TC1, TC2) obtained in the first threshold validation step (541) from neighboring sensing unit (15_{i,j}) are within a predefined further variability range (S′), wherein the second threshold validation step (542) compares the first and/or second threshold values (TA1, TA2, TC1, TC2) obtained in the first threshold validation step (541) from neighboring sensing unit (15_{i,j}) one with another and if the first and/or second threshold values (TA1, TA2, TC1, TC2) obtained in the first threshold validation step (541) are within a predefined further variability range (S′) it is provided for calculating and storing in the memory unit (24) a mean value of the first and/or second threshold values (TA1, TA2, TC1, TC2), the second threshold validation step (542) being performed after the first threshold validation step (541) and only on the first and/or second threshold values (TA1, TA2, TC1, TC2) obtained by the first threshold validation step (541).

16. (canceled)

17. (canceled)

18. (canceled)

19. (canceled)

20. The method according to claim 1, further comprising a fault check process (55) for determining the similarity in calibrated data dc(i,j,t) from different sensing units close one to another and for evaluating if one or more sensing units (15) and/or one or more layers (12) of the sensing device (1) are faulty, and to provide fault-check data (df(i,j,t)) from the sensing unit which are not considered faulty.

21. The method according to claim 20, wherein the fault check process (55) comprises a fault calculation phase (55A) in which it is calculated in the fault check time (Tf) the coherence (C) between the calibrated data dc(i,j,t) in each fault check window Fwi of a plurality of fault check windows (Fwᵢ, i=1, 2, . . . , Nwf) of the fault check time (Tf) from each pair of sensing units of the plurality of sensing units (10, 15_{i,j}) is calculated so as to obtain a coherence matrix (Cab) and comparing the values of the coherence matrix (Cab) with a fault threshold value (TFa) for deciding if sensing unit (15a,j) is possibly faulty.

22. (canceled)

23. The method according to claim 1, further comprising a gait segmentation process (80) in which it is provided for defining a plurality of gait phases of the task performed by the user, the gait segmentation process (80) comprising a calculation phase (81) in which calibrated data (dc(i,j,t)) from the at least one sensing unit (15_{i,j}) are received and evaluated and a gait evaluation phase (82) in which the data obtained in the gait calculation phase (81) are sent and displayed indicating the gait phase and/or stored in the memory unit (24).

24. The method according to claim 23, wherein the gait segmentation process (80) comprises creating at least set of predefined weight matrices (MwL) for each condition of use, each matrix (Mwl) of the set of weight matrices (MwL) indicating the supposed level of activation of each sensing unit (15_{i,j}) in each phase (I) of the condition of use.

25. The method according to claim 24, wherein the gait segmentation process (80) comprises multiplying at preset time (t*) each matrix (Mwl) of the set of weight matrices (MwL) of the condition of use for the current calibrated value (dc(i,j, t*)) from each sensing unit (15i,j), and defining the actual gait phase (I) as the phase (I) for which it is obtained the maximum value of the multiplication.

26. Sensing device (1) for sensing pressure exerted by a portion of the body and/or the human prosthesis, the sensing device (1) comprising a body (11) formed by a flexible material and having a plurality of superimposed layers (12), each layer of the plurality of layers being provided with a plurality of sensing units (10) arranged into the body (11) and configured for sensing the pressure applied by the portion of the body of the user and/or the human prosthesis, each sensing units (15_{i,j}) of the plurality of sensing units (10) being a capacitive or resistive/capacitive force/pressure sensing unit.

27. The sensing device (1) according to claim 26, wherein the sensing units (15i,j) of the plurality of sensing units (10) are so positioned to define in the body (11) at least one inert region (14) spacing the sensing units (15).

28. Sensing device (1) according to claim 26, wherein the sensing units (15_{i,j}) of the different layers (12ᵢ) are positioned in corresponding positions in the body (11) of the sensing device (1).

29-32. (canceled)

33. Acquisition device (2) for an analysis system (100) arranged for analysing analyzing pressure exerted by a portion of the body and/or of a human prosthesis, the acquisition device (2) comprising: connection means (3) for connecting the acquisition device (2) to a sensing device (1, 10, 15_{i,j}) arranged for being positioned in contact with the portion of the body and/or the human prosthesis to be analyzed and configured for measuring generated data generated by the portion of the body and/or the human prosthesis, and for transferring emitted data to the acquisition device, the acquisition device (2) comprising a processing device (23) for sampling data generated by the portion of the body and/or the human prosthesis during a threshold calibration period at a sampling frequency in at least one condition of use so as to obtain an array of sampled data d(i,j,t) of the at least one sensing unit (15i,j), a memory unit (24) in which at least one threshold value (TA1, TC1) of the sampled data d(i,j,t) for the at least one condition of use is stored and the processing device (23) being further suitable for comparing the sampled data (d(i,j,t)) with the at least one threshold value (TA1, TC1) to obtain calibrated data (dc(i,j,t)) and to store the calibrated data (dc(i,j,t)) in a memory unit and/or to send calibrated data (dc(i,j,t)) to a computing device if they are greater than the stored at least one threshold values (TA1, TC1).

34-37. (canceled)
